(19) United States
(12) Patent Application Publication
(10) Pub. No.: US 2024/0216470 A1
(43) Pub. Date: Jul. 4, 2024

(54) INDUCIBLE IL-2 AND PD-1/PD-L1 COMBINATION THERAPY

(71) Applicants: Werewolf Therapeutics, Inc., Watertown, MA (US); MSD International Business GmbH, Luzern (CH)

(72) Inventors: William WINSTON, West Newton, MA (US); Daniel HICKLIN, Boston, MA (US); Jose Andres SALMERON-GARCIA, Acton, MA (US); Cynthia SEIDEL-DUGAN, Belmont, MA (US); Heather BRODKIN, West Newton, MA (US); Randi ISAACS, East Hampton, NY (US)

(21) Appl. No.: 18/440,775

(22) Filed: Feb. 13, 2024

Related U.S. Application Data
(63) Continuation of application No. PCT/US2022/040485, filed on Aug. 16, 2022.
(60) Provisional application No. 63/233,966, filed on Aug. 17, 2021.

(51) Int. Cl. A61K 38/20; A61K 31/337; A61K 31/4439; A61K 31/47; A61K 31/513; A61K 31/519; A61K 39/00; A61K 39/395; A61K 45/06; A61P 35/00

(52) U.S. Cl. CPC A61K 38/2013; A61K 31/337; A61K 31/4439; A61K 31/47; A61K 31/513; A61K 31/519; A61K 39/3955; A61K 45/06; A61P 35/00; A61K 2039/545

(57) ABSTRACT

This disclosure relates to methods and compositions for treating cancer using a combination therapy comprising an inducible IL-2 prodrug in combination with pembrolizumab.

### CROSS REFERENCE TO RELATED APPLICATIONS

This application claims the benefit of U.S. Provisional Application No. 63/233,966, filed on Aug. 17, 2021, the disclosure of which is hereby incorporated by reference in its entirety.

### FIELD OF THE INVENTION

Disclosed herein are therapies useful for the treatment of cancer and methods for treating cancer, which comprises administering a combination of an anti-PD-1 antibody, or antigen binding fragment thereof, and an inducible IL-2 prodrug.

### BACKGROUND

Interleukin-2 (IL-2) has potent immunostimulatory activity and can be effective in eradicating tumors in mouse models and a rectableombinant IL-2 therapy (aldesleukin) was approved by the US FDA for treatment of metastatic renal cell carcinoma and metastatic melanoma. Aldesleukin has demonstrated complete cancer regression in about 10% of patients treated for metastatic melanoma and renal cancer. Unfortunately, rIL-2 has poor pharmacokinetic (PK) properties and dose-limiting systemic toxicities due to binding to the high and medium affinity IL-2 receptors (IL-2Rα/β/γ and IL-2Rβ/γ, respectively) in the periphery. High-dose IL-2 administration results in severe hypotension and vascular leak syndrome (VLS), which has relegated its use to specialized care centers and limited its dosing to reach efficacious levels. (Pachella LA, et al. JAcdv Pract Oncol. 2015; 6(3):212-221.) These side effects limit the number of patients who can tolerate the recommended therapeutic regimen and, consequently, achieve the full clinical benefit from IL-2 therapy.

Inducible forms of IL-2, that are conditionally activated in the tumor microenvironment through protease cleavage to release the fully active, native IL-2 cytokine within the tumor to stimulate a potent anti-tumor immune response, are described in WO2021097376. These IL-2 prodrugs include a native IL-2 molecule attached through a protease cleavable linker to a half-life extension domain (e.g., anti-human serum albumin antibody binding fragment such as a VH domain) and an IL-2 blocking element (e.g., anti-IL-2 antibody binding fragment, such as a Fab) to block binding of IL-2 to IL-2β/γ receptors on normal tissue in the periphery. Upon cleavage of the protease cleavable linker, fully active native IL-2 is released within the tumor to stimulate a potent anti-tumor immune response.

Immune checkpoint inhibitors are proteins that regulate T cell functions. T cell effector function is important for immunotherapeutic approaches to treating tumors. But immunosuppression, and decreased effector function, is often seen as tumors grow and cancer progresses. One mechanism behind this phenomenon is the activation of immune checkpoint inhibitors by cancer cells, leading to suppression of the anti-tumor immune response. This typically occurs when cancer cells express proteins on their surface that can interact with immune checkpoint proteins on the surface of T cells in the tumor microenvironment to suppress the activity of the T cells. Immune checkpoint proteins include, for example, PD-1 which binds ligands PD-L1 (B7-H1, CD274) and PD-L2 (B7-DC, CD273), CTLA-4 (CD152) which binds B7-1 (CD80) and B7-2 (CD86), LAG 3 (CD223) which binds Galectin3, LSECtin and FGL1; TIM3 (HAVCR2) which binds ligands Ceacaml and Galectin9; TIGIT (VSTM3, WUCAM) which binds CD112 and CD155; BTLA (CD272) which binds HVEM (TNFRSF14), B7-H3 (CD276), B7-H4 (VTCN1), VISTA (B7-H5), KIR, CD44 (2B4), CD160 (BY55) which bind HVEM; CD134 (TNRFSR4, OX40) which binds CD252 (OX-40L).

PD-1 is recognized as an important player in immune regulation and the maintenance of peripheral tolerance. PD-1 is moderately expressed on naive T, B and NKT cells and up-regulated by T/B cell receptor signaling on lymphocytes, monocytes and myeloid cells (Sharpe et al., The function of programmed cell death 1 and its ligands in regulating autoimmunity and infection. Nature Immunology (2007); 8:239-245).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (Dong, Haidong et al., Tumor-associated B7-H1 promotes T-cell apoptosis: a potential mechanism of immune evasion. Nat Med. 2002 Aug.;8(8):793-800; Yang, Wanhua et al., PD-1 interaction contributes to the functional suppression of T-cell responses to human uveal melanoma cells in vitro. Invest Ophthalmol Vis Sci. 2008 June; 49(6 (2008): 49: 2518-2525; Ghebeh, Hazem et al., The B7-H1 (PD-L1) T lymphocyte-inhibitory molecule is expressed in breast cancer patients with infiltrating ductal carcinoma: correlation with important high-risk prognostic factors. Neoplasia (2006) 8: 190-198; Hamanishi, Junzo et al., Programmed cell death 1 ligand 1 and tumor-infiltrating CD8+T lymphocytes are prognostic factors of human ovarian cancer. Proc. Natl. Acad. Sci. USA (2007): 104: 3360-3365; Thompson, R Houston, and Eugene D Kwon, Significance of B7-H1 overexpression in kidney cancer. Clinical genitourin Cancer (2006): 5: 206-211; Nomi, Takeo et al., Clinical significance and therapeutic potential of the programmed death-1 ligand/programmed death-1 pathway in human pancreatic cancer. Clinical Cancer Research (2007);13:2151-2157; Ohigashi, Yuichiro et al., Clinical significance of programmed death-1 ligand-1 and programmed death-1 ligand 2 expression in human esophageal cancer. Clin. Cancer Research (2005): 11: 2947-2953; Inman, Brant A et al., PD-L1 (B7-H1) expression by urothelial carcinoma of the bladder and BCG-induced granulomata: associations with localized stage progression. Cancer (2007): 109: 1499-1505; Shimauchi, Takatoshi et al., Augmented expression of programmed death-1 in both neoplasmatic and nonneoplastic CD4+ T-cells in adult T-cell Leukemia/Lymphoma. Int. J. Cancer (2007): 121:2585-2590; Gao, Qiang et al., Overexpression of PD-L1 significantly associates with tumor aggressiveness and postoperative recurrence in human hepatocellular carcinoma. Clinical Cancer Research (2009) 15: 971-979; Nakanishi, Juro et al., Overexpression of B7-H1 (PD-L1) significantly associates with tumor grade and postoperative prognosis in human urothelial cancers. Cancer Immunol Immunother. (2007) 56: 1173-1182; Hino et al., Tumor cell expression of programmed cell death-1 is a prognostic factor for malignant melanoma. Cancer (2010): 00: 1-9).

Similarly, PD-1 expression on tumor infiltrating lymphocytes was found to mark dysfunctional T cells in breast cancer and melanoma (Ghebeh, Hazem et al., Foxp3+ tregs and B7-H1+/PD-1+T lymphocytes co-infiltrate the tumor tissues of high-risk breast cancer patients: implication for immunotherapy. BMC Cancer. 2008 Feb. 23; 8:57; Ahmadzadeh, Mojgan et al., Tumor antigen-specific CD8 T cells infiltrating the tumor express high levels of PD-1 and are functionally impaired. Blood (2009) 114: 1537-1544) and to correlate with poor prognosis in renal cancer (Thompson, R Houston et al., PD-1 is expressed by tumor infiltrating cells and is associated with poor outcome for patients with renal carcinoma. Clinical Cancer Research (2007) 15: 1757-1761). Thus, it has been proposed that PD-L1-expressing tumor cells interact with PD-1-expressing T cells to attenuate T cell activation and evasion of immune surveillance, thereby contributing to an impaired immune response against the tumor.

Immune checkpoint therapies targeting the PD-1 axis have resulted in groundbreaking improvements in clinical response in multiple human cancers (Brahmer et al., N Engl J Med 2012, 366: 2455-65; Garon et al. N Engl J Med 2015, 372: 2018-28; Hamid et al., N Engl J Med 2013, 369: 134-44; Robert et al., Lancet 2014, 384: 1109-17; Robert et al., N Engl J Med 2015, 372: 2521-32; Robert et al., N Engl J Med 2015, 372: 320-30; Topalian et al., N Engl J Med 2012, 366: 2443-54; Topalian et al., J Clin Oncol 2014, 32: 1020-30; Wolchok et al., N Engl J Med 2013, 369: 122-33).

Therapeutic agents, such as antibodies, that bind immune checkpoint proteins and inhibit their immunosuppressive activity have been developed as anti-tumor agents. Several such agents are now commercially available for cancer therapy, including the anti-PD1 antibodies pembrolizumab (KEYTRUDA™), Merck and Co., Inc., Rahway, NJ, USA, dostarlimab (JEMPERLI), cemiplimab-rwlc (LIBATYO), nivolumab (OPDIVO™), Bristol-Myers Squibb Company, Princeton, NJ, USA), camrelizumab, tislelizumab, toripalimab, and sintilimab (TYVYT); the anti-PD-L1 antibodies avelumab (BAVENCIO), durvalumab (IMFINZI), and atezolizumab (TECENTRIQ); the anti-CTLA-4 antibody ipilimumab (YERVOY). While therapy with such immune checkpoint inhibitors provide advantages for cancer therapy, the overall success remains low, relapse occurs and resistance to checkpoint inhibition develops. There is an unmet medical need for improved methods for treating cancer.

### SUMMARY

This disclosure relates to compositions and methods for treating cancer using an inducible IL-2 prodrug and an anti-PD-1 antibody, such as pembrolizumab. The method generally comprises administering to a subject in need thereof an effective amount of an inducible IL-2 prodrug and an anti-PD-1 antibody or antigen binding fragment thereof. A preferred anti-PD-1 antibody is pembrolizumab. The inducible IL-2 prodrug can be Compound 1, Compound 2, Compound 3, or Compound 4.

The inducible IL-2 prodrug is conditionally active. The inducible IL-2 prodrug comprises two polypeptide chains. The first polypeptide chain comprises from amino to carboxy terminus: the IL-2 polypeptide—a protease cleavable linker—an anti-human serum albumin (HSA) binding single antibody variable domain—a linker that is preferably protease cleavable—VH and CH1 of an antibody that binds IL-2. The second polypeptide chain comprises a VL and CL of an antibody that binds IL-2 and that together with the VH and CH1 of the first polypeptide chain form a Fab that binds the IL-2 polypeptide. When the inducible IL-2 prodrug is not in a site of interest (e.g., a tumor microenvironment), the prodrug typically remains intact. The intact prodrug has attenuated IL-2 receptor agonist activity. When the inducible IL-2 prodrug is in a site of interest (such as a tumor microenvironment), the protease cleavable linker is cleaved by a protease active in the site of interest, releasing an unattenuated form of IL-2. This conditional activity preserves the immune stimulatory effects of IL-2 while limiting the systemic toxicity associated with non-inducible IL-2 therapy. The intact IL-2 prodrug contains an element that extends its half-life, but the post-cleavage unattenuated form of IL-2 does not. As a result, the short half-life of IL-2 effectively limits toxicity outside of the site of interest.

Without being bound by theory, the immune stimulatory effects of the inducible IL-2 prodrug and the prevention of immune suppression caused by an anti-PD-1 antibody or an antigen binding fragment may allow the subject's immune system to more effectively combat cancer.

Described herein are methods for treating cancer comprises administering to a subject in need thereof an a combination therapy comprising Compound 1, Compound 2, Compound 3, Compound 4 or an amino acid sequence variant of any of the foregoing and an anti-PD-1 antibody, or antigen binding fragment thereof; wherein the anti-PD-1 antibody, or antigen binding fragment thereof, comprises: (a) light chain CDRs SEQ ID NOs: 6, 7 and 8 and (b) heavy chain CDRs SEQ ID NOs: 11, 12 and 13. The methods disclosed herein comprise administering to a subject in need thereof an effective amount of the combination therapy disclosed herein.

Compound 1 comprises a first polypeptide chain of SEQ ID NO:1 and a second polypeptide chain of SEQ ID NO:5, and the amino acid sequence variant of Compound 1 can comprise a first polypeptide chain that has at least about 80% identity to SEQ ID NO:1 and a second polypeptide chain can comprise at least about 80% identity to SEQ ID NO:5. Compound 2 comprises a first polypeptide chain of SEQ ID NO:2 and a second polypeptide chain of SEQ ID NO:5, and the amino acid sequence variant of Compound 2 can comprise a first polypeptide chain that has at least about 80% identity to SEQ ID NO:2 and a second polypeptide chain that has at least about 80% identity to SEQ ID NO:5. Compound 3 comprises a first polypeptide chain of SEQ ID NO:3 and a second polypeptide chain of SEQ ID NO:5, and the amino acid sequence variant of Compound 3 can comprise a first polypeptide chain that has at least about 80% identity to SEQ ID NO:3 and a second polypeptide chain that has at least about 80% identity to SEQ ID NO:5. Compound 4 comprises a first polypeptide chain of SEQ ID NO:1 and a second polypeptide chain of SEQ ID NO:4, and the amino acid sequence variant of Compound 4 can comprise a first polypeptide chain that has at least about 80% identity to SEQ ID NO:4 and a second polypeptide chain that has at least about 80% identity to SEQ ID NO:5.

The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain comprising the amino acid sequences in SEQ ID NO: 10 and a light chain comprising the amino acid sequence in SEQ ID NO: 5. The anti-PD-1 antibody can be pembrolizumab, a pembrolizumab variant or a biosimilar.

Compound 1, Compound 2, Compound 3, Compound 4 or an amino acid sequence variant can be administered before, concurrently with or after the anti-PD-1 antibody, or antigen binding fragment thereof. For example, the anti-PD-1 antibody, or antigen binding fragment thereof, can be administered, and then about 30 minutes following completion of the administration of the anti-PD-1 antibody, or antigen binding fragment thereof, Compound 1, Compound 2, Compound 3, Compound 4 or an amino acid sequence variant can be administered.

Compound 1, Compound 2, Compound 3, Compound 4 or an amino acid sequence variant of any of the foregoing and the anti-PD-1 antibody, or antigen binding fragment thereof can be administered intravenously. For example, administration can be by an intravenous infusion. Other administration routes include, but are not limited to, oral, parenteral, intravenous, intra-articular, intraperitoneal, intramuscular, subcutaneous, intracavity, transdermal, intrahepatical, intracranial, nebulization/inhalation, by installation via bronchoscopy, or intratumoral.

About 100 mg to about 600 mg of the anti-PD-1 antibody or antigen binding fragment thereof can be administered about every three to six weeks during the course of therapy. In instances, the patient is administered 200 mg, 240 mg, 400 mg, 480 mg, 720 mg, or 2 mg/kg of the anti-PD-1 antibody. In instances, 200 mg of the anti-PD-1 antibody or antigen binding fragment thereof can be administered about every three weeks during the course of therapy. In some instances, 400 mg of the anti-PD1 antibody or antigen binding fragment thereof can be administered about every six weeks during the course of therapy. In instances, the human patient is administered 200 mg pembrolizumab once every three weeks. In instances, the human patient is administered 2 mg/kg pembrolizumab once every three weeks. In instances, the human patient is administered 400 mg pembrolizumab once every six weeks.

About 1 mg to about 500 mg of Compound 1, Compound 2, Compound 3, Compound 4 or an amino acid sequence variant of any of the foregoing can be administered every two weeks during the course of therapy. For instance, 1 mg to about 240 mg of Compound 1, Compound 2, Compound 3, Compound 4 or an amino acid sequence variant of any of the foregoing can be administered every two weeks during the course of therapy. For instance, 1 mg, about 3 mg, about 10 mg, about 30 mg, about 60 mg, about 120 mg or about 240 mg of Compound 1, Compound 2, Compound 3, Compound 4 or an amino acid sequence variant of any of the foregoing can be administered every two weeks during the course of therapy.

The combination can be administered as a first line therapy. The subject suitable for the methods disclosed herein may have failed to achieve a complete response to a prior treatment or to an ongoing treatment. An example of prior treatment or ongoing treatment can comprise treatment with a checkpoint inhibitor. The checkpoint inhibitor can be an anti-PD-1 antibody or an anti-PD-L1 antibody. The checkpoint inhibitor can be pembrolizumab.

The checkpoint inhibitor can be an anti-CTLA-4 antibody.

Cancers suitable for treatment according to the methods disclosed herein include, but are not limited to, adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In particular embodiments, the cancer can be melanoma, non-small cell lung cancer (NSCLC), small cell lung cancer (SCLC), head and neck squamous cell cancer (HNSCC), classical Hodgkin lymphoma (cHL), primary mediastinal large B cell lymphoma (PMBCL), urothelial carcinoma, microsatellite instability high or mismatch repair deficient cancer, microsatellite instability high or mismatch repair deficient colorectal cancer, gastric cancer, esophageal cancer, cervical cancer, hepatocellular carcinoma (HCC), Merkel cell carcinoma (MCC), renal cell carcinoma (RCC), endometrial carcinoma, tumor mutational burden high cancer, cutaneous squamous cell carcinoma (cSCC), triple negative breast cancer (TNBC), or colorectal cancer.

In particular embodiments, the cancer is selected from the group consisting of: melanoma, non-small cell lung cancer, head and neck squamous cell cancer, classical Hodgkin lymphoma, primary mediastinal large B-cell lymphoma, urothelial carcinoma, microsatellite instability-high or mismatch repair deficient cancer, gastric cancer, esophageal cancer, cervical cancer, hepatocellular carcinoma, Merkel cell carcinoma, renal cell carcinoma, endometrial carcinoma, a cancer characterized by a tumor having a high mutational burden, cutaneous squamous cell carcinoma, and triple negative breast cancer.

The cancer can be metastatic, a solid tumor, a sarcoma or carcinoma. The cancer can be colon cancer, lung cancer, melanoma, renal cell carcinoma, or breast cancer. The cancer can be metastatic renal clear cell carcinoma or metastatic cutaneous malignant melanoma.

The cancer can be non-small cell lung cancer, and the method can further comprise administering to the subject pemetrexed and a platinum chemotherapeutic agent. For example, the chemotherapeutic agent can be paclitaxel or protein bound paclitaxel. The cancer can be head and neck squamous cell cancer, and the method can further comprise administering to the subject fluorouracil. The cancer can be esophageal or gastroesophageal junction carcinoma, and the method can further comprise administering to the subject a platinum- and fluoropyrimidine-based chemotherapeutic agent. The cancer can be a renal cell carcinoma.

The cancer can be endometrial carcinoma. The cancer can be triple-negative breast cancer, and the method can further comprise administering to the subject a chemotherapeutic agent.

The disclosure also relates to pharmaceutical compositions comprising an inducible IL-2 prodrug in combination with a PD-1 anatagonist such as pembrolizumab and pembrolizumab variants. The pharmaceutical composition can be used to treat cancer according to the methods disclosed herein. The pharmaceutical composition can be a liquid composition for intravenous administration. Alternatively, the composition can be a lyophilized composition. The lyophilized composition can be reconstituted using water for injection or saline and the reconstituted formulation can be suitable for intravenous administration.

### BRIEF DESCRIPTION OF THE DRAWINGS

FIG. 1 is graph showing average tumor volume progression in mice implanted with a MC38 tumor and treated with different dose levels of Compound 1, an IL-2 prodrug with a non-cleavable linker (Comp. 1-NC), or only vehicle.

FIG. 2 is a chart showing the amounts of tumor specific CD8+ T cells in spleens removed from animals 40 days after MC38 tumor implantation. The animals were treated with an inducible IL-2 prodrug or only vehicle. Tetramer staining was used to identify tumor specific CD8+ T cells, and those cells were examined for memory markers.

FIG. 3 is a graph showing tumor volume progression in naive mice or mice that previously had complete MC38 tumor regressions following treatment with an inducible IL-2 prodrug. Mice were rechallenged with MC38 tumor cells 60 days after the initial implantation. No treatment was administered during the rechallenge.

FIG. 4 is a series of charts showing cell counts of various immune cell types in tumors extracted from mice were implanted with MC38 tumors and treated with either an inducible IL-2 prodrug or only vehicle.

FIG. 5 is a series of graphs showing intact inducible IL-2 prodrug and free IL-2 levels in plasma and tumor samples collected at various timepoints after dosing mice bearing MC38 tumors with an inducible IL-2 prodrug.

FIG. 6 is a series of graphs showing plasma exposure of Compound 1 or rIL-2 after non human primates were dosed with increasing amounts of Compound 1 from 0.05 mg/kg to 1.25 mg/kg once per week for 2 weeks.

FIG. 7A is a graph showing tumor volume progression in a B16F10 tumor model in which mice were treated with 100 μg of Compound 1, 200 μg of Compound 1, or only vehicle. 1×10⁵ tumor cells were implanted in the flank of animals and tumor growth was monitored. Once tumors reached an average volume of 30-60 mm³, the animals were randomized and dosed. Tumor volumes and body weights were recorded three times per week.

FIG. 7B is a graph showing tumor volume progression in a B16F10 tumor model in which mice were treated with 100 μg of Compound 1, 200 μg of anti-PD-1 antibody (RMP1-14) alone, 100 μg of Compound 1 and 200 μg of anti-PD-1 antibody (RMP1-14), or only vehicle. 1×10⁵ tumor cells were implanted in the flank of animals and tumor growth was monitored. Once tumors reached an average volume of 30-60 mm³, the animals were randomized and dosed. Tumor volumes and body weights were recorded three times per week.

FIG. 8 is a graph showing body weight progression in a B16F10 tumor model in which mice were treated with Compound 1, Compound 1 and RMP1-14, or only vehicle. 1×10⁵ tumor cells were implanted in the flank of animals and tumor growth was monitored. Once tumors reached an average volume of 30-60 mm³, the animals were randomized and dosed. Body weights were recorded three times per week.

FIG. 9 is a study design schematic for the clinical trial described in Example 2.

FIG. 10A is a graph showing average tumor volumes in mice treated with vehicle (PBS, circles), inducible IL-2 prodrug (Compound 1, squares), anti-PD-1 (triangles), or inducible IL-2 prodrug (Compound 1) and anti-PD-1 (diamonds). The dosing schedule is also shown (arrows).

FIGS. 10B-10E are graphs of tumor volume in individual mice treated with vehicle (FIG. 10B), inducible IL-2 prodrug (Compound 1, FIG. 10C), anti-PD-1 (FIG. 10D), or inducible IL-2 prodrug (Compound 1) and anti-PD-1 (FIG. 10E).

FIG. 10F is a graph showing average body weights of mice treated with vehicle (PBS, circles), inducible IL-2 prodrug (Compound 1, squares), anti-PD-1 (triangles), and inducible IL-2 prodrug and anti-PD-1 (diamonds).

### DETAILED DESCRIPTION

This disclosure relates to methods and compositions for treating cancer using a combination therapy comprising an inducible IL-2 prodrug in combination with a PD-1 anatagonist such as pembrolizumab and pembrolizumab variants.

### Definitions and Abbreviations

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless the context requires otherwise. Furthermore, use of the term “including” as well as other forms, such as “include”, “includes,” and “included,” is not limiting.

The section headings used herein are for organizational purposes only and are not to be construed as limiting the subject matter described.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions. “Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

### Inducible IL-2 Prodrug

The inducible IL-2 prodrug for use in the methods and compositions of this disclosure overcome the toxicity and short half-life problems that have severely limited the clinical use of cytokines in oncology. The inducible IL-2 prodrug contains an IL-2 polypeptide that has receptor agonist activity of native IL-2, including binding to and activating signaling through IL-2Rα/β/γ and IL-2Rβ/γ, but in the context of the inducible pro-drug, the cytokine receptor agonist activity is attenuated, and the circulating half-life is extended. The prodrug includes protease cleavage sequences, which are cleaved by proteases that are associated with, and are typically enriched or selectively present in, the tumor microenvironment. Thus, the inducible IL-2 prodrugs are preferentially (or selectively) and efficiently cleaved in the tumor microenvironment to release active IL-2, and to limit IL-2 activity substantially to the tumor microenvironment. The IL-2 that is released upon cleavage has a short half-life, which is substantially similar to the half-life of naturally occurring IL-2, further restricting IL-2 activity to the tumor microenvironment. Even though the half-life of the inducible IL-2 prodrug is extended, toxicity is dramatically reduced or eliminated because the circulating prodrug has attenuated IL-2 activity, and active IL-2 is restricted to the tumor microenvironment.

The inducible IL-2 prodrug comprises two polypeptide chains. The first polypeptide chain comprises from amino to carboxy terminus: the IL-2 polypeptide—a protease cleavable linker—an anti-human serum albumin (HSA) binding single antibody variable domain—a linker that is preferably protease cleavable—VH and CH1 of an antibody that binds IL-2. The second polypeptide chain comprises a VL and CL of an antibody that binds IL-2 and that together with the VH and CH1 of the first polypeptide chain form a Fab that binds the IL-2 polypeptide. Compounds 1, 2, 3 and 4 are specific examples of inducible IL-2 prodrugs for use according to this disclosure. Compounds 1, 2, 3, and 4 and additional details regarding their activity is disclosed in WO2021/097376.

TABLE 1
Inducible IL-2 prodrugs

| IL-2 Prodrug | First Polypeptide | Second Polypeptide |
|---|---|---|
| Compound 1 | SEQ ID NO: 1 | SEQ ID NO: 5 |
| Compound 2 | SEQ ID NO: 2 | SEQ ID NO: 5 |
| Compound 3 | SEQ ID NO: 3 | SEQ ID NO: 5 |
| Compound 4 | SEQ ID NO: 4 | SEQ ID NO: 5 |

Amino acid sequence variants of compounds 1, 2, 3 and 4, that retain attenuated IL-2 activity in the periphery and that release active IL-2 upon protease cleavage in the tumor microenvironment can also be used in accordance with this disclosure. For example, a prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO:1 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO:5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO:2 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO:5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO:3 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO:5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO:4 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO:5.

For all amino acid sequence variant prodrugs, it is preferred that the protease cleavage site contain no amino acid replacements, or only conservative amino acid replacements, so that the sequence variant prodrug is cleaved in the tumor microenvironment and releases IL-2 to substantially the same degree as the corresponding parental prodrug. Similarly, it is preferred that the complementarity determining regions of the anti-HAS single variable domain and the anti-IL2 Fab contain no amino acid replacements, or only conservative amino acid replacements, so that a) the serum half-life of the sequence variant prodrug is substantially the same as the corresponding parental prodrug, and b) the attenuation of IL-2 agonist activity of the sequence variant prodrug is substantially the same as the corresponding parental prodrug.

Exemplary amino acid substitutions are provided in Table 2.

TABLE 2
Exemplary amino acid substitutions

| | Exemplary |
|---|---|
| Amino Acid | Substitutions |
| Ala | Ser, Gly, Cys |
| Arg | Lys, Gln, Met, Ile |
| Asn | Gln, His, Glu, Asp |
| Asp | Glu, Asn, Gln |
| Cys | Ser, Met, Thr |
| Gln | Asn, Lys, Glu, Asp |
| Glu | Asp, Asn, Gln |
| Gly | Pro, Ala |
| His | Asn, Gln |
| Ile | Leu, Val, Met |
| Leu | Ile, Val, Met |
| Lys | Arg, Gln, Met, Ile |
| Met | Leu, Ile, Val |
| Phe | Met, Leu, Tyr, Trp, His |
| Ser | Thr, Met, Cys |
| Thr | Ser, Met, Val |
| Trp | Tyr, Phe |
| Tyr | Trp, Phe, His |
| Val | Ile, Leu, Met |

### PD-1 Antagonists

The word “pembrolizumab” is an international nonproprietary name that refers to the humanized anti-PD-1 antibody marketed under the brand name “Keytruda” by Merck & Co., Inc. (Rahway, NJ, USA) Pembrolizumab (formerly known as MK-3475, SCH 900475 and lambrolizumab) alternatively referred to herein as “pembro,” is a humanized IgG4 mAb with the structure described in WHO Drug Information, Vol. 27, No. 2, pages 161-162 (2013) and which comprises the heavy and light chain amino acid sequences and CDRs described in Table 3. Pembrolizumab has been approved by the U.S. FDA as described in the Prescribing Information for KEYTRUDA™ (Merck & Co., Inc., Rahway, NJ USA; initial U.S. approval 2014, updated June 2022).

A “PD-1 antagonist” or “Anti-PD-1 antibody” as used in the any of the treatment methods, means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCDIL1, PDL1, B7H1, B7-4, CD274 and B7-H for PD-L1; and PDCD1L2, PDL2, B7-DC, Btdc and CD273 for PD-L2. In any of the treatment methods, medicaments and uses of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists useful in the treatment methods, medicaments and uses of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in some embodiments, the human constant region is an IgG1 or IgG4 constant region. In some embodiments, the antigen binding fragment is selected from the group consisting of Fab, Fab′-SH, F(ab′)₂, scFv and Fv fragments.

A specific anti-human PD-1 mAbs useful as the PD-1 antagonist in the treatment method, medicaments and uses of the present invention is pembrolizumab. Pembrolizumab is a potent humanized IgG4 monoclonal antibody with high specificity of binding to the programmed cell death 1 (PD-1) receptor, thus inhibiting its interaction with programmed cell death ligand 1 (PD-L1) and programmed cell death ligand 2 (PD-L2). PD-1 ligands are expressed at high levels in some tumors and signaling through the PD-1/PD-1 ligand pathway can contribute to inactivation of T cell immune surveillance of tumors. Pembrolizumab binds to PD-1 and blocks it interaction with PD-L1 and PD-L2, releasing PD-1 pathway-mediated inhibition of the immune response, including the anti-tumor immune response. Pembrolizumab is indicated for the treatment of patients across a number of cancer indications including melanoma, non-small cell lung cancer (NSCLC), small cell lung cancer (SCLC), head and neck squamous cell cancer (HNSCC), classical Hodgkin lymphoma (cHL), primary mediastinal large B cell lymphoma (PMBCL), urothelial carcinoma, microsatellite instability high or mismatch repair deficient cancer, microsatellite instability high or mismatch repair deficient colorectal cancer, gastric cancer, esophageal cancer, cervical cancer, hepatocellular carcinoma (HCC), Merkel cell carcinoma (MCC), renal cell carcinoma (RCC), endometrial carcinoma, tumor mutational burden high cancer, cutaneous squamous cell carcinoma (cSCC) and triple negative breast cancer (TNBC). The heavy and light chain amino acid sequences of pembrolizumab are shown in Table 3. See, U.S. Pat. Nos. 8,354,509 and 8,900,587, both of which are incorporated herein by reference in their entirety.

A variant or biosimilar of pembrolizumab may also be used in the treatment method, medicaments and uses of the present invention. As used herein, a “pembrolizumab variant” means a monoclonal antibody which comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g, the variant positions are located in the FR regions or the constant region, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

In some embodiments of the treatment methods, medicaments and uses of the present invention, the PD-1 antagonist is a monoclonal antibody, or antigen binding fragment thereof, which comprises: (a) light chain CDRs SEQ ID NOs: 6, 7 and 8 and (b) heavy chain CDRs SEQ ID NOs: 11, 12 and 13.

In some embodiments of the treatment methods, medicaments and uses of the present invention, the PD-1 antagonist is a monoclonal antibody, or antigen binding fragment thereof, which comprises (a) a heavy chain variable region comprising SEQ ID NO:14 or a variant thereof, and (b) a light chain variable region comprising SEQ ID NO:9 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

In another embodiment of the treatment methods, medicaments and uses of the present invention, the PD-1 antagonist is a monoclonal antibody comprising (a) a heavy chain comprising SEQ ID NO: 15 and (b) a light chain comprising SEQ ID NO:19.

In all of the treatment methods, medicaments and uses of the present invention, the PD-1 antagonist inhibits the binding of PD-L1 to PD-1, and preferably also inhibits the binding of PD-L2 to PD-1. In some embodiments of the above treatment method, medicaments and uses, the PD-1 antagonist is a monoclonal antibody, or an antigen binding fragment thereof, which specifically binds to PD-1 and blocks the binding of PD-L1 to PD-1. In one embodiment, the PD-1 antagonist is an anti-PD-1 antibody, or antigen binding fragment thereof, which comprises: (a) light chain CDRs SEQ ID NOs: 6, 7 and 8 and (b) heavy chain CDRs SEQ ID NOs: 11, 12 and 13. In one embodiment, the PD-1 antagonist is an anti-PD-1 antibody, or antigen binding fragment thereof, which comprises (a) a heavy chain variable region comprising SEQ ID NO:14 or a variant thereof, and (b) a light chain variable region comprising SEQ ID NO:9 or a variant thereof. In one embodiment, the PD-1 antagonist is an anti-PD-1 antibody which comprises a heavy chain and a light chain, and wherein the heavy and light chains comprise the amino acid sequences in SEQ ID NO:10 and SEQ ID NO:5, respectively. In one embodiment, the PD-1 antagonist is pembrolizumab or a pembrolizumab variant. In one embodiment, the PD-1 anatagonist is pembrolizumab.

Table 3 below provides a list of the amino acid sequences of exemplary anti-PD-1 antibodies for use in the treatment method, medicaments and uses of the present invention.

TABLE 3
Exemplary Anti-PD-1 antibody Sequences

| | | |
|---|---|---|
| Antibody | | SEQ ID |
| Feature | Amino Acid Sequence | NO. |

Pembrolizumab Light Chain
(Comprises light chain CDRs, light chain, and variable light chain of hPD-1.09A in
WO2008/156712)

| | | |
|---|---|---|
| CDR1 | RASKGVSTSGYSYLH | 6 |
| CDR2 | LASYLES | 7 |
| CDR3 | QHSRDLPLT | 8 |
| Variable | EIVLTQSPATLSLSPGERATLSCRASKGVSTSGYSYLHWYQQKPGQAP | 9 |
| Region | RLLIYLASYLESGVPARFSGSGSGTDFTLTISSLEPEDFAVYYCQHSRD | |
| | LPLTFGGGTKVEIK | |
| Light Chain | EIVLTQSPATLSLSPGERATLSCRASKGVSTSGYSYLHWYQQKPGQAP | 10 |
| | RLLIYLASYLESGVPARFSGSGSGTDFTLTISSLEPEDFAVYYCQHSRD | |
| | LPLTFGGGTKVEIKRTVAAPSVFIFPPSDEQLKSGTASVVCLLNNFYPR | |
| | EAKVQWKVDNALQSGNSQESVTEQDSKDSTYSLSSTLTLSKADYEK | |
| | HKVYACEVTHQGLSSPVTKSFNRGEC | |

Pembrolizumab Heavy Chain
(Comprises heavy chain CDRs, heavy chain, and variable heavy chain of hPD-1.09A in
WO2008/156712)

| | | |
|---|---|---|
| CDR1 | NYYMY | 11 |
| CDR2 | GINPSNGGTNFNEKFKN | 12 |
| CDR3 | RDYRFDMGFDY | 13 |
| Variable | QVQLVQSGVEVKKPGASVKVSCKASGYTFTNYYMYWVRQAPGQGL | 14 |
| Region | EWMGGINPSNGGTNFNEKFKNRVTLTTDSSTTTAYMELKSLQFDDT | |
| | AVYYCARRDYRFDMGFDYWGQGTTVTVSS | |
| Heavy Chain | QVQLVQSGVEVKKPGASVKVSCKASGYTFTNYYMYWVRQAPGQGL | 15 |
| | EWMGGINPSNGGTNFNEKFKNRVTLTTDSSTTTAYMELKSLQFDDT | |
| | AVYYCARRDYRFDMGFDYWGQGTTVTVSSASTKGPSVFPLAPCSRS | |
| | TSESTAALGCLVKDYFPEPVTVSWNSGALTSGVHTFPAVLQSSGLYS | |
| | LSSVVTVPSSSLGTKTYTCNVDHKPSNTKVDKRVESKYGPPCPPCPAP | |
| | EFLGGPSVFLFPPKPKDTLMISRTPEVTCVVVDVSQEDPEVQFNWYV | |
| | DGVEVHNAKTKPREEQFNSTYRVVSVLTVLHQDWLNGKEYKCKVS | |
| | NKGLPSSIEKTISKAKGQPREPQVYTLPPSQEEMTKNQVSLTCLVKGF | |
| | YPSDIAVEWESNGQPENNYKTTPPVLDSDGSFFLYSRLTVDKSRWQE | |
| | GNVFSCSVMHEALHNHYTQKSLSLSLGK | |

%

### Combination Therapy and Pharmaceutical Compositions

This disclosure further relates to methods for treating cancer using a combination therapy comprising an inducible IL-2 prodrug in combination with a PD-1 antagonist (e.g., an anti-PD-1 antibody, e.g., pembrolizumab). This disclosure also relates to pharmaceutical compositions for use in such methods, compositions which may also be referred to as medicaments. This disclosure provides pharmaceutical compositions comprising a PD-1 antagonist for use in combination with an inducible IL-2 prodrug, pharmaceutical compositions comprising an inducible IL-2 prodrug for use in combination with a PD-1 antagonist, and pharmaceutical compositions that contain an inducible IL-2 prodrug in combination with the PD-1 antagonist, preferably prembrolizumab or a prembrolizumab variant.

The combination therapy may also comprise one or more additional therapeutic agents. The additional therapeutic agent may be, e.g., a chemotherapeutic, a biotherapeutic agent, an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF). The specific dosage and dosage schedule of the additional therapeutic agent can further vary, and the optimal dose, dosing schedule and route of administration will be determined based upon the specific therapeutic agent that is being used.

The disclosure relates to methods for treating cancer comprising administering to a subject in need thereof a combination therapy that includes an inducible IL-2 prodrug and a PD-1 antagonist such as pembrolizumab or a pembrolizumab variant. The inducible IL-2 prodrug and the PD-1 antagonist, such as prembrolizumab are administered to the subject so that there is overlap of the pharmacological activities of the two therapeutic agents. Accordingly, the inducible IL-2 prodrug can be administered before, after, concurrently, or periprocedurally with the PD-1 antagonist, such as pembrolizumab. In some practices of the methods, the PD-1 antagonist, such as pembrolizumab is administered before the inducible IL-2 prodrug. In some practices of the methods, the PD-1 antagonist, such as pembrolizumab, is administered after the inducible IL-2 prodrug. In some particular practices of the methods, the PD-1 antagonist, such as pembrolizumab is administered, then about 30 minutes after the PD-1 antagonist, such as pembrolizumab administration is completed, the inducible IL-2 prodrug is administered.

The term “effective amount,” as used herein, refers to the amount of agent (inducible IL-2 prodrug and PD-1 antagonist) that is administered to achieve the desired effect under the conditions of administration, such an amount that reduces tumor size, reduces tumor burden, extends progression free survival or extends overall survival. The actual effective amount selected will depend on the particular cancer being treated and its stage and other factors, such as the subject's age, gender, weight, ethnicity, prior treatments and response to those treatments and other factors. Suitable amounts of inducible IL-2 prodrug and PD-1 antagonist, such as pembrolizumab to be administered, and dosage schedules for a particular patient can be determined by a clinician of ordinary skill based on these and other considerations.

For example, about 1 mg to about 500 mg of inducible IL-2 prodrug can be administered about every two weeks; and about 100 mg to about 600 mg of pembrolizumab or a pembrolizumab variant can be administered about every three to six weeks (e.g., 200 mg every three weeks (Q3W) or 400 mg every six weeks (Q6W)). For example, about 1 mg to about 500 mg of inducible IL-2 prodrug can be administered about every two weeks; and 200 mg or 400 mg of pembrolizumab or a pembrolizumab variant can be administered about every three to six weeks (e.g., 200 mg every three weeks (Q3W) or 400 mg every six weeks (Q6W)). Pediatric dosing can vary. For example, for pediatric patients, pembrolizumab is typically administered at about 2 mg/kg, up to 200 mg, every three weeks.

The methods and compositions of this disclosure can be administered to a subject in need thereof as first line therapy or subsequent to or in addition to other therapies. The subject in need of therapy according to this disclosure can be a subject who failed to achieve a complete response to prior treatment or ongoing treatment, or failed to achieve a partial response to prior treatment or ongoing treatment. For example, the subject in need or therapy according to this disclosure can be a subject who failed to achieve a complete response, or a partial response, to prior or ongoing therapy that includes a checkpoint inhibitor. The subject in need of therapy according to this disclosure can be a subject who failed to achieve stable disease for longer than 6 months (SD>6) due to ongoing or prior therapy that included a checkpoint inhibitor. In embodiments, the subject failed to achieve a complete response, or a partial response, to prior or ongoing therapy with 1) an anti-PD1 antibody, such as pembrolizumab (KEYTRUDA), dostarlimab (JEMPERLI), cemiplimab-rwlc (LIBATYO), nivolumab (OPDIVO), camrelizumab, tislelizumab, toripalimab, and sintilimab (TYVYT); 2) and anti-PD-L1 antibody, such as avelumab (BAVENCIO), durvalumab (IMFINZI), and atezolizumab (TECENTRIQ); or 3) an anti-CTLA-4 antibody, such as ipilimumab (YERVOY).

The inducible IL-2 prodrug and the PD-1 antagonist (e.g., pembrolizumab or a pembrolizumab variant) are typically administered systemically, for example by intravenous injection or preferably intravenous infusion. Other types of administration can be used, such as orally, parenterally, intravenously, intra-articularly, intraperitoneally, intramuscularly, subcutaneously, intracavity, transdermally, intrahepatically, intracranially, nebulization/inhalation, by installation via bronchoscopy, or intratumorally. In some embodiments, the PD-1 antagonist is administered subcutaneously.

In certain embodiments of the invention, the PD-1 antagonist in the combination therapy is an anti-PD-1 antibody, or antigen binding fragment thereof, as described herein. The anti-PD-1 antibody, or antigen binding fragment thereof, can be administered in a liquid medicament at a dose selected from the group consisting of 1 mg/kg Q2W, 2 mg/kg Q2W, 3 mg/kg Q2W, 5 mg/kg Q2W, 10 mg/kg Q2W, 1 mg/kg Q3W, 2 mg/kg Q3W, 3 mg/kg Q3W, 5 mg/kg Q3W, 10 mg/kg Q3W and flat-dose equivalents of any of these doses, i.e., such as 200 mg Q3W or 400 mg Q6W. In some embodiments, anti-PD-1 antibody, or antigen binding fragment thereof is provided as a liquid medicament which comprises 25 mg/ml of the anti-PD-1 antibody, or antigen binding fragment thereof, 7% (w/v) sucrose, 0.02% (w/v) polysorbate 80 in 10 mM histidine buffer pH 5.5. In other embodiments, the anti-PD-1 antibody, or antigen binding fragment thereof, is provided as a liquid medicament which comprises about 125 to about 200 mg/mL of the anti-PD-1 antibody, or antigen binding fragment thereof, about 10 mM histidine buffer; about 10 mM L-methionine, or a pharmaceutically acceptable salt thereof; about 7% (w/v) sucrose; and about 0.02% (w/v) polysorbate 80. In one embodiment, the anti-PD-1 antibody, or antigen binding fragment thereof, comprises: (a) light chain CDRs SEQ ID NOs: 6, 7 and 8 and (b) heavy chain CDRs SEQ ID NOs: 11, 12 and 13. In one embodiment, the anti-PD-1 antibody, or antigen binding fragment thereof, comprises (a) a heavy chain variable region comprising SEQ ID NO:14 or a variant thereof, and (b) a light chain variable region comprising SEQ ID NO:9 or a variant thereof. In one embodiment, the anti-PD-1 antibody comprises a heavy chain and a light chain, and wherein the heavy and light chains comprise the amino acid sequences in SEQ ID NO:10 and SEQ ID NO:5, respectively. In one embodiment, the anti-PD-1 antibody is pembrolizumab or a pembrolizumab variant. In one embodiment, the anti-PD-1 antibody is pembrolizumab.

In some embodiments, the selected dose of the anti-PD-1 antibody, or antigen binding fragment thereof, is administered by IV infusion. In one embodiment, the selected dose of the anti-PD-1 antibody, or antigen binding fragment thereof, is administered by IV infusion over a time period of between 25 and 40 minutes, or about 30 minutes. In other embodiments, the selected dose of the anti-PD-1 antibody, or antigen binding fragment thereof, is administered subcutaneously. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO:10 and SEQ ID NO:5, respectively. The anti-PD-1 antibody can be pembrolizumab or a pembrolizumab variant. The anti-PD-1 antibody can be pembrolizumab.

In some embodiments, the patient is treated with the combination therapy for at least 24 weeks, e.g., eight 3-week cycles. In some embodiments, treatment with the combination therapy continues until the patient exhibits evidence of PD effect, or a CR, or progressive disease.

The methods and compositions disclosed herein can be used to treat any suitable cancer, in particular solid tumors, such as sarcomas and carcinomas. For examples, the methods and compositions disclosed herein can be used to treat adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, mycosis fungoides, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, non-Hodgkin lymphoma, squamous carcinoma of the head and neck, malignant pleural mesothelioma, and Wilms tumor.

Preferably, the methods and compositions disclosed herein are used to treat colon cancer, lung cancer, melanoma, renal cell carcinoma, or breast cancer.

In certain embodiments, the methods and compositions disclosed herein are used to treat melanoma, non-small cell lung cancer (NSCLC), small cell lung cancer (SCLC), head and neck squamous cell cancer (HNSCC), classical Hodgkin lymphoma (cHL), primary mediastinal large B cell lymphoma (PMBCL), urothelial carcinoma, microsatellite instability high or mismatch repair deficient cancer, microsatellite instability high or mismatch repair deficient colorectal cancer, gastric cancer, esophageal cancer, cervical cancer, hepatocellular carcinoma (HCC), Merkel cell carcinoma (MCC), renal cell carcinoma (RCC), endometrial carcinoma, tumor mutational burden high cancer, cutaneous squamous cell carcinoma (cSCC), triple negative breast cancer (TNBC), urothelial carcinoma, colorectal cancer or oesophageal carcinoma.

Any of the methods and compositions disclosed herein can be used to treat a human subject who has a a cancer that tests positive for one or both of PD-L1 and PD-L2, and preferably tests positive for PD-L1 expression. In some embodiments, PD-L1 expression is detected using a diagnostic anti-human PD-L1 antibody, or antigen binding fragment thereof, in an IHC assay on an FFPE or frozen tissue section of a tumor sample removed from the patient. Typically, the subject's physician would order a diagnostic test to determine PD-L1 expression in a tumor tissue sample removed from the patient prior to initiation of treatment with the PD-1 antagonist but it is envisioned that the physician could order the first or subsequent diagnostic tests at any time after initiation of treatment, such as for example after completion of a treatment cycle. In one embodiment, the PD-L1 expression is measured by the PD-L1 IHC 22C3 pharmDx assay. In another embodiment, the patient has a Mononuclear Inflammatory Density Score for PD-L1 expression≥2. In another embodiment, the subject has a Mononuclear Inflammatory Density Score for PD-L1 expression 3. In another embodiment, the subject has a Mononuclear Inflammatory Density Score for PD-L1 expression≥4. In another embodiment, the subject has a Tumor Proportion Score for PD-L1 expression 1%. In another embodiment, the subject has a Tumor Proportion Score for PD-L1 expression 10%. In another embodiment, the subject has a Tumor Proportion Score for PD-L1 expression≥20%. In another embodiment, the subject has a Tumor Proportion Score for PD-L1 expression≥30%. In another embodiment, the subject has a Tumor Proportion Score for PD-L1 expression 40%. In another embodiment, the subject has a Tumor Proportion Score for PD-L1 expression 50%. In a further embodiment, the subject has a Combined Positive Score for PD-L1 expression≥1%. In a further embodiment, the subject has a Combined Positive Score for PD-L1 expression between 1 and 20%. In a further embodiment, the subject has a Combined Positive Score for PD-L1 expression≥2%. In a further embodiment, the subject has a Combined Positive Score for PD-L1 expression≥5%. In yet a further embodiment, the subject has a Combined Positive Score for PD-L1 expression≥10%. In a further embodiment, the subject has a Combined Positive Score for PD-L1 expression≥15%. In yet a further embodiment, the subject has a Combined Positive Score for PD-L1 expression≥20%.

In certain embodiments, the methods and compositions disclosed herein are used to treat melanoma. As an example, the methods and compositions disclosed herein can be used to treat melanoma in subjects with unresectable or metastatic melanoma. As another example, the methods and compositions disclosed herein can be used for the adjuvant treatment of subjects with melanoma with involvement of lymph node(s) following complete resection.

In certain embodiments, the methods and compositions disclosed herein are used to treat non-small cell lung cancer (NSCLC). As an example, the methods and compositions disclosed herein can be used to treat NSCLC in subjects with NSCLC expressing PD-L1 (e.g., Tumor Proportion Score (TPS)≥1%) as determined by an FDA-approved test, with no EGFR or ALK genomic tumor aberrations, and is: stage III where subjects are not candidates for surgical resection or definitive chemoradiation, or metastatic. As another example, the methods and compositions disclosed herein can be used to treat NSCLC in patients with metastatic NSCLC whose tumors express PD-L1 (TPS≥1%) as determined by an FDA-approved test, with disease progression on or after platinum-containing chemotherapy. As another example, the methods and compositions disclosed herein can be used in combination with pemetrexed and platinum chemotherapy, as first-line treatment of patients with metastatic nonsquamous NSCLC, with no EGFR or ALK genomic tumor aberrations. As another example, the methods and compositions disclosed herein can be used in combination with carboplatin and either paclitaxel or paclitaxel protein-bound, as first-line treatment of patients with metastatic squamous NSCLC.

In certain embodiments, the methods and compositions disclosed herein are used to treat SCLC. As an example, the methods and compositions disclosed herein can be used to treat SCLC in subjects with metastatic SCLC with disease progression on or after platinum-based chemotherapy and at least one other prior line of therapy.

In certain embodiments, the methods and compositions disclosed herein are used to treat HNSCC. As an example, the methods and compositions disclosed herein can be used to treat HNSCC in subjects with metastatic or with unresectable, recurrent HNSCC whose tumors express PD-L1 (e.g., Combined Positive Score (CPS)≥1) as determined by an FDA-approved test. As another example, the methods and compositions disclosed herein can be used to treat HNSCC in subjects with recurrent or metastatic HNSCC with disease progression on or after platinum-containing chemotherapy. As another example, the methods and compositions disclosed herein can be used in combination with platinum and fluorouracil for the first-line treatment of patients with metastatic or with unresectable, recurrent HNSCC.

In certain embodiments, the methods and compositions disclosed herein are used to treat cHL. As an example, the methods and compositions disclosed herein can be used to treat cHL in subjects with relapsed or refractory cHL. As another example, the methods and compositions disclosed herein can be used to treat cHL in pediatric subjects with refractory cHL, or cHL that has relapsed after 2 or more lines of therapy.

In certain embodiments, the methods and compositions disclosed herein are used to treat PMBCL. As an example, the methods and compositions disclosed herein can be used to treat PMBCL in subjects with refractory PMBCL, or in subjects who have relapsed after 2 or more prior lines of therapy.

In certain embodiments, the methods and compositions disclosed herein are used to treat urothelial carcinoma. As an example, the methods and compositions disclosed herein can be used to treat urothelial carcinoma in subjects with locally advanced or metastatic urothelial carcinoma who are not eligible for cisplatin-containing chemotherapy and whose tumors express PD-L1 (e.g., Combined Positive Score (CPS)≥10) as determined by an FDA-approved test, or in subjects who are not eligible for any platinum-containing chemotherapy regardless of PD-L1 status. As another example, the methods and compositions disclosed herein can be used to treat urothelial carcinoma in subjects with locally advanced or metastatic urothelial carcinoma who have disease progression during or following platinum-containing chemotherapy or within 12 months of neoadjuvant or adjuvant treatment with platinum-containing chemotherapy. As another example, the methods and compositions disclosed herein can be used to treat urothelial carcinoma in subjects with Bacillus Calmette-Guerin (BCG)-unresponsive, high-risk, non-muscle invasive bladder cancer (NMIBC) with carcinoma in situ (CIS) with or without papillary tumors who are ineligible for or have elected not to undergo cystectomy.

In certain embodiments, the methods and compositions disclosed herein are used to treat Microsatellite Instability-High (MSI-H) or Mismatch Repair Deficient (dMMR) Cancer. As an example, the methods and compositions disclosed herein can be used to treat MSI-H or dMMR cancer in subjects with unresectable or metastatic MSI-H or dMMR cancer wherein the solid tumors have progressed following prior treatment and the subject has no satisfactory alternative treatment options, or wherein the colorectal cancer has progressed following treatment with a fluoropyrimidine, oxaliplatin, and irinotecan.

In certain embodiments, the methods and compositions disclosed herein are used to treat Microsatellite Instability-High (MSI-H) or Mismatch Repair Deficient (dMMR) Colorectal Cancer. As an example, the methods and compositions disclosed herein can be used to treat MSI-H or dMMR colorectal cancer in subjects with unresectable or metastatic MSI-H or dMMR colorectal cancer.

In certain embodiments, the methods and compositions disclosed herein are used to treat gastric cancer. As an example, the methods and compositions disclosed herein can be used to treat gastric cancer in subjects with recurrent locally advanced or metastatic gastric or gastroesophageal junction adenocarcinoma whose tumors express PD-L1 (e.g., Combined Positive Score (CPS)≥1) as determined by an FDA-approved test, with disease progression on or after 2 or more prior lines of therapy including fluoropyrimidine- and platinum-containing chemotherapy and if appropriate, HER2/neu-targeted therapy.

In certain embodiments, the methods and compositions disclosed herein are used to treat esophageal cancer. As an example, the methods and compositions disclosed herein can be used to treat esophageal cancer in subjects with locally advanced or metastatic esophageal or gastroesophageal junction (GEJ) (e.g., tumors with epicenter 1 to 5 centimeters above the GEJ) carcinoma that is not amenable to surgical resection or definitive chemoradiation, in combination with platinum- and fluoropyrimidine-based chemotherapy. As another example, the methods and compositions disclosed herein can be used to treat esophageal cancer in subjects with locally advanced or metastatic esophageal or gastroesophageal junction (GEJ) (e.g., tumors with epicenter 1 to 5 centimeters above the GEJ) carcinoma that is not amenable to surgical resection or definitive chemoradiation, after one or more prior lines of systemic therapy for patients with tumors of squamous cell histology that express PD-L1 (CPS≥10) as determined by an FDA-approved test.

In certain embodiments, the methods and compositions disclosed herein are used to treat cervical cancer. As an example, the methods and compositions disclosed herein can be used to treat cervical cancer in subjects with recurrent or metastatic cervical cancer with disease progression on or after chemotherapy whose tumors express PD-L1 (e.g., Combined Positive Score (CPS)≥1) as determined by an FDA-approved test.

In certain embodiments, the methods and compositions disclosed herein are used to treat HCC. As an example, the methods and compositions disclosed herein can be used to treat HCC in subjects who have been previously treated with sorafenib.

In certain embodiments, the methods and compositions disclosed herein are used to treat MCC. As an example, the methods and compositions disclosed herein can be used to treat MCC in subjects with recurrent locally advanced or metastatic MCC.

In certain embodiments, the methods and compositions disclosed herein are used to treat RCC. As an example, the methods and compositions disclosed herein can be used in combination with axitinib, for the first-line treatment of patients with advanced RCC.

In certain embodiments, the methods and compositions disclosed herein are used to treat endometrial carcinoma. As an example, the methods and compositions disclosed herein can be used in combination with lenvatinib, for the treatment of subjects with advanced endometrial carcinoma that is not MSI-H or dMMR, who have disease progression following prior systemic therapy and are not candidates for curative surgery or radiation.

In certain embodiments, the methods and compositions disclosed herein are used to treat Tumor Mutational Burden-High (TMB-H) Cancer. As an example, the methods and compositions disclosed herein can be used to treat TMB-H cancer in subjects with unresectable or metastatic tumor mutational burden-high (e.g., ≥10 mutations/megabase (mut/Mb)) solid tumors, as determined by an FDA-approved test, that have progressed following prior treatment and who have no satisfactory alternative treatment options.

In certain embodiments, the methods and compositions disclosed herein are used to treat Cutaneous Squamous Cell Carcinoma (cSCC). As an example, the methods and compositions disclosed herein can be used to treat cSCC in subjects with recurrent or metastatic cutaneous squamous cell carcinoma that is not curable by surgery or radiation.

In certain embodiments, the methods and compositions disclosed herein are used to treat Triple-Negative Breast Cancer (TNBC). As an example, the methods and compositions disclosed herein can be used in combination with chemotherapy, for the treatment of subjects with locally recurrent unresectable or metastatic TNBC whose tumors express PD-L1 (e.g., Combined Positive Score (CPS)≥10) as determined by an FDA approved test.

The cancer to be treated using the methods and compositions of this disclosure can be metastatic cancer.

In certain embodiments, the methods and compositions disclosed herein are used to treat metastatic renal clear cell carcinoma or metastatic cutaneous malignant melanoma.

In embodiments of the method, compound 1, 2, 3 or 4 or an amino acid sequence variant thereof is administered to the subject with cancer in an amount of about 1 mg to about 240 mg every three weeks, and pembrolizumab (or a pembrolizumab variant or biosimilar) is administered in an amount of 200 mg about every three weeks (Q3W) or 400 mg about every six weeks (Q6W).

Compound 1, 2, 3 or 4 or an amino acid sequence variant thereof can be administered to the subject with cancer in an amount of about 1 mg every three weeks, and an anti-PD-1 antibody, or antigen binding fragment thereof is administered in an amount of 200 mg about every three weeks or 400 mg about every six weeks to treat cancer or a tumor as disclosed here, such as metastatic renal clear cell carcinoma or metastatic cutaneous malignant melanoma. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO:10 and SEQ ID NO:5, respectively. The anti-PD-1 antibody can be pembrolizumab or a pembrolizumab variant. The anti-PD-1 antibody can be pembrolizumab (or a biosimilar).

Compound 1, 2, 3 or 4 or an amino acid sequence variant thereof can be administered to the subject with cancer in an amount of about 1 mg to about 3 mg (e.g. 1 mg, 2 mg or 3 mg) every three weeks, and an anti-PD-1 antibody, such as pembrolizumab, or antigen binding fragment thereof, is administered in an amount of 200 mg about every three weeks (Q3W) or 400 mg about every six weeks (Q6W) to treat cancer or a tumor as disclosed here, such as metastatic renal clear cell carcinoma or metastatic cutaneous malignant melanoma. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO:10 and SEQ ID NO:5, respectively. The anti-PD-1 antibody can be pembrolizumab or a pembrolizumab variant. The anti-PD-1 antibody can be pembrolizumab (or a biosimilar).

Compound 1, 2, 3 or 4 or an amino acid sequence variant thereof can be administered to the subject with cancer in an amount of about 10 mg every three weeks, and an anti-PD-1 antibody, such as pembrolizumab, or antigen binding fragment thereof, is administered in an amount of 200 mg about every three weeks or 400 mg about every six weeks to treat cancer or a tumor as disclosed here, such as metastatic renal clear cell carcinoma or metastatic cutaneous malignant melanoma. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO:10 and SEQ ID NO:5, respectively. The anti-PD-1 antibody can be pembrolizumab or a pembrolizumab variant. The anti-PD-1 antibody can be pembrolizumab (or a biosimilar).

Compound 1, 2, 3 or 4 or an amino acid sequence variant thereof can be administered to the subject with cancer in an amount of about 30 mg every three weeks, and an anti-PD-1 antibody, such as pembrolizumab, or antigen binding fragment thereof, is administered in an amount of 200 mg every about three weeks or 400 mg about every six weeks to treat cancer or a tumor as disclosed here, such as metastatic renal clear cell carcinoma or metastatic cutaneous malignant melanoma. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO:10 and SEQ ID NO:5, respectively. The anti-PD-1 antibody can be pembrolizumab or a pembrolizumab variant. The anti-PD-1 antibody can be pembrolizumab (or a biosimilar).

Compound 1, 2, 3 or 4 or an amino acid sequence variant thereof can be administered to the subject with cancer in an amount of about 60 mg every three weeks, and an anti-PD-1 antibody, or antigen binding fragment thereof, is administered in an amount of 200 mg about every three weeks or 400 mg about every six weeks to treat cancer or a tumor as disclosed here, such as metastatic renal clear cell carcinoma or metastatic cutaneous malignant melanoma. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO:10 and SEQ ID NO:5, respectively. The anti-PD-1 antibody can be pembrolizumab or a pembrolizumab variant. The anti-PD-1 antibody can be pembrolizumab (or a biosimilar).

Compound 1, 2, 3 or 4 or an amino acid sequence variant thereof can be administered to the subject with cancer in an amount of about 120 mg every three weeks, and an anti-PD-1 antibody, or antigen binding fragment thereof, is administered in an amount of 200 mg about every three weeks or 400 mg about every six weeks to treat cancer or a tumor as disclosed here, such as metastatic renal clear cell carcinoma or metastatic cutaneous malignant melanoma. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO:10 and SEQ ID NO:5, respectively. The anti-PD-1 antibody can be pembrolizumab or a pembrolizumab variant. The anti-PD-1 antibody can be pembrolizumab (or a biosimilar).

Compound 1, 2, 3 or 4 or an amino acid sequence variant thereof can be administered to the subject with cancer in an amount of about 240 mg every three weeks, and an anti-PD-1 antibody, or antigen binding fragment thereof, is administered in an amount of 200 mg about every three weeks or 400 mg about every six weeks to treat cancer or a tumor as disclosed here, such as metastatic renal clear cell carcinoma or metastatic cutaneous malignant melanoma. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO:10 and SEQ ID NO:5, respectively. The anti-PD-1 antibody can be pembrolizumab or a pembrolizumab variant. The anti-PD-1 antibody can be pembrolizumab (or a biosimilar).

If desired, additional therapeutic agents can be administered to the subject. Typically such additional therapeutic agents are anti-cancer agents such as chemotherapeutic agents (e.g., adriamycin, cerubidine, bleomycin, alkeran, velban, oncovin, fluorouracil, thiotepa, methotrexate, bisantrene, noantrone, thiguanine, cytaribine, procarabizine), other immunocheck point inhibitors (e.g., anti-CTLA4 (ipilimumab (YERVOY)), other cytokines (such as IL-12 or inducible IL-12 prodrugs), angiogenesis inhibitors, antibody-drug conjugates (e.g., trastuzumab emtansine (KADCYLA), trastuzumab deruxtecan (ENHERTU), enfortumab vedotin (PADCEV), sacituzumab govitecan (TRODELVY), cellular therapies (e.g., CAR-T, TCT-T, T-cell therapy, such as tumor infiltrating lymphocyte (TIL) therapy), oncolytic viruses, radiation therapy and/or small molecules.

An additional therapeutic agent can be, for example, pemetrexed, a platinum chemotherapeutic agent, carboplatin, paclitaxel, protein-bound paclitaxel, fluorouracil, a fluoropyrimidine-based chemotherapeutic agent, or axitinib.

For example, a method for treating metastatic nonsquamous non-small cell lung cancer can comprise administering an inducible IL-2 prodrug as disclosed herein, an anti-PD-1 antibody (or antigen binding fragment thereof), pemetrexed and a platinum chemotherapeutic agent. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO:10 and SEQ ID NO:5, respectively. The anti-PD-1 antibody can be pembrolizumab or a pembrolizumab variant. The anti-PD-1 antibody can be pembrolizumab (or a biosimilar).

In another example, a method for treating metastatic squamous non-small cell lung cancer can comprise administering inducible IL-2 prodrug as disclosed herein, an anti-PD-1 antibody (or antigen binding fragment thereof), and paclitaxel. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO: 10 and SEQ ID NO:5, respectively. The anti-PD-1 antibody can be pembrolizumab or a pembrolizumab variant. The anti-PD-1 antibody can be pembrolizumab (or a biosimilar).

In another example, a method for treating metastatic squamous non-small cell lung cancer can comprise administering inducible IL-2 prodrug as disclosed herein, an anti-PD-1 antibody (or antigen binding fragment thereof), and protein bound paclitaxel. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO:10 and SEQ ID NO:5, respectively. The anti-PD-1 antibody can be pembrolizumab or a pembrolizumab variant. The anti-PD-1 antibody can be pembrolizumab (or a biosimilar).

In another example, a method for treating metastatic or with unresectable, recurrent head and neck squamous cell cancer can comprise administering inducible IL-2 prodrug as disclosed herein, an anti-PD-1 antibody (or antigen binding fragment thereof), and fluorouracil. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO:10 and SEQ ID NO:5, respectively. The anti-PD-1 antibody can be pembrolizumab or a pembrolizumab variant. The anti-PD-1 antibody can be pembrolizumab (or a biosimilar).

In another example, a method for treating locally advanced or metastatic esophageal or gastroesophageal junction carcinoma that is not amenable to surgical resection or definitive chemoradiation comprises administering inducible IL-2 prodrug as disclosed herein, an anti-PD-1 antibody (or antigen binding fragment thereof), and a platinum- and fluoropyrimidine-based chemotherapeutic agent. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO:10 and SEQ ID NO:5, respectively. The anti-PD-1 antibody can be pembrolizumab or a pembrolizumab variant. The anti-PD-1 antibody can be pembrolizumab (or a biosimilar).

In another example, a method for treating advanced renal cell carcinoma comprises administering inducible IL-2 prodrug as disclosed herein, an anti-PD-1 antibody (or antigen binding fragment thereof), and axitinib. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO: 10 and SEQ ID NO:5, respectively. The anti-PD-1 antibody can be pembrolizumab or a pembrolizumab variant. The anti-PD-1 antibody can be pembrolizumab (or a biosimilar).

In another example, a method for treating renal cell carcinoma comprises administering compound 1, 2, 3 or 4 or an amino acid sequence variant thereof can be administered to the subject with cancer in an amount of about 240 mg every three weeks, and an anti-PD-1 antibody, or antigen binding fragment thereof, is administered in an amount of 200 mg about every three weeks or 400 mg about every six weeks, and axitinib administered in an amount of 5 mg orally twice daily. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO:10 and SEQ ID NO:5, respectively. The anti-PD-1 antibody can be pembrolizumab or a pembrolizumab variant. The anti-PD-1 antibody can be pembrolizumab (or a biosimilar).

In another example, a method for treating advanced endometrial carcinoma that is not microsatellite instability-high or mismatch repair deficient, in subjects who have disease progression following prior systemic therapy and are not candidates for curative surgery or radiation, comprises administering inducible IL-2 prodrug as disclosed herein, an anti-PD-1 antibody (or antigen binding fragment thereof), and lenvatinib. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO: 10 and SEQ ID NO:5, respectively. The anti-PD-1 antibody can be pembrolizumab or a pembrolizumab variant. The anti-PD-1 antibody can be pembrolizumab (or a biosimilar).

In another example, a method for treating endometrial carcinoma or renal cell carcinoma comprises administering compound 1, 2, 3 or 4 or an amino acid sequence variant thereof can be administered to the subject with cancer in an amount of about 240 mg every three weeks, and an anti-PD-1 antibody, or antigen binding fragment thereof, is administered in an amount of 200 mg about every three weeks or 400 mg about every six weeks, and lenvatinib administered in an amount of 20 mg orally once daily. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO:10 and SEQ ID NO:5, respectively. The anti-PD-1 antibody can be pembrolizumab or a pembrolizumab variant. The anti-PD-1 antibody can be pembrolizumab (or a biosimilar).

In another example, a method for treating locally recurrent unresectable or metastatic triple-negative breast cancer whose tumors express PD-L1 comprises administering inducible IL-2 prodrug as disclosed herein, an anti-PD-1 antibody (or antigen binding fragment thereof), and a chemotherapeutic agent. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO:10 and SEQ ID NO:5, respectively. The anti-PD-1 antibody can be pembrolizumab or a pembrolizumab variant. The anti-PD-1 antibody can be pembrolizumab (or a biosimilar).

The pharmaceutical compositions can take a variety of forms, e.g., liquid, lyophilized, and typically contain a suitable pharmaceutically acceptable carrier. Pharmaceutically acceptable carriers (or excipients) are the non-active ingredient components of the pharmaceutical composition and are not biologically or otherwise undesirable, i.e., the material is administered to a subject without causing undesirable biological effects or interacting in a deleterious manner with the other components of the pharmaceutical formulation or composition in which it is contained. Carriers are frequently selected to minimize degradation of the active ingredient and to minimize adverse side effects in the subject.

Suitable carriers and their formulations are described in Remington: The Science and Practice of Pharmacy, 21^{st} Edition, David B. Troy, ed., Lippicott Williams & Wilkins (2005). Examples of the pharmaceutically-acceptable carriers include, but are not limited to, sterile water, saline, buffered solutions like Ringer's solution, and dextrose solution. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the chimeric polypeptides or nucleic acid sequences encoding the chimeric polypeptides to humans or other subjects.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like. Typically, an appropriate amount of a pharmaceutically-acceptable salt is used in the formulation to render the formulation isotonic, although the formulation can be hypertonic or hypotonic if desired. The pH of the solution is generally about 5 to about 8 or from about 7 to 7.5.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable.

This disclosure also relates to a kit that includes a) a pharmaceutical composition that contains an inducible IL-2 prodrug composition, for example as a liquid composition or a lyophilized composition, in a suitable container (e.g., a vial, bag or the like), and b) a composition comprising an anti-PD-1 antibody (or antigen binding fragment thereof), for example as a liquid composition or a lyophilized composition, in a suitable container (e.g., a vial, bag or the like). The kit can further include other components, such as sterile water or saline for reconstitution of lyophilized compositions. The anti-PD-1 antibody, or antigen binding fragment thereof, can comprise: (a) light chain CDRs SEQ ID NOs: 6, 7 and 8 and (b) heavy chain CDRs SEQ ID NOs: 11, 12 and 13. The anti-PD-1 antibody, or antigen binding fragment thereof, can comprise (a) a heavy chain variable region comprising SEQ ID NO:14 or a variant thereof, and (b) a light chain variable region comprising SEQ ID NO:9 or a variant thereof. The anti-PD-1 antibody can comprise a heavy chain and a light chain, and wherein the heavy and light chains comprise the amino acid sequences in SEQ ID NO:10 and SEQ ID NO:5, respectively. The anti-PD-1 antibody can be pembrolizumab or a pembrolizumab variant. The anti-PD-1 antibody can be pembrolizumab (or a biosimilar).

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

“Cytokine” is a well-known term of art that refers to any of a class of immunoregulatory proteins (such as interleukin or interferon) that are secreted by cells especially of the immune system and that are modulators of the immune system. Cytokine polypeptides that can be used in the fusion proteins disclosed herein include, but are not limited to transforming growth factors, such as TGF-α and TGF-β (e.g., TGFbeta1, TGFbeta2, TGFbeta3); interferons, such as interferon-α, interferon-β, interferon-γ, interferon-kappa and interferon-omega; interleukins, such as IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21 and IL-25; tumor necrosis factors, such as tumor necrosis factor alpha and lymphotoxin; chemokines (e.g., C—X—C motif chemokine 10 (CXCL10), CCL19, CCL20, CCL21), and granulocyte macrophage-colony stimulating factor (GM-CS), as well as fragments of such polypeptides that active the cognate receptors for the cytokine (i.e., functional fragments of the foregoing). “Chemokine” is a term of art that refers to any of a family of small cytokines with the ability to induce directed chemotaxis in nearby responsive cells.

As used herein, the terms “inducible” refer to the ability of a protein, i.e. IL-2, that is part of a prodrug, to bind its receptor and effectuate activity upon cleavage of the prodrug in the tumor microenvironment. The inducible IL-2 prodrugs disclosed herein have attenuated or no IL-2 agonist activity, but upon cleavage in the tumor microenvironment release active IL-2.

“Attenuated” activity, means that biological activity and typically IL-2 receptor agonist activity is decreased as compared to the activity of natural IL-2. The inducible IL-2 prodrugs disclosed herein have attenuated IL-2 receptor agonists activity, that is at least about 10×, at least about 50×, at least about 100×, at least about 250×, at least about 500×, at least about 1000×or less agonist activity as compared to natural IL-2. Upon cleavage in the tumor microenvironment, IL-2 is release that is active. Typically, the IL-2 that is released has IL-2 receptor agonist activity that is at least about 10×, at least about 50×, at least about 100×, at least about 250×, at least about 500×, or at least about 1000×greater than the IL-2 receptor activating activity of the prodrug.

As used herein, the terms “peptide”, “polypeptide”, or “protein” are used broadly to mean two or more amino acids linked by a peptide bond. Protein, peptide, and polypeptide are also used herein interchangeably to refer to amino acid sequences. It should be recognized that the term polypeptide is not used herein to suggest a particular size or number of amino acids comprising the molecule and that a peptide of the invention can contain up to several amino acid residues or more.

As used throughout, “subject” can be a vertebrate, more specifically a mammal (e.g. a human, horse, cat, dog, cow, pig, sheep, goat, mouse, rabbit, rat, and guinea pig), birds, reptiles, amphibians, fish, and any other animal. In some embodiments, the mammal is a human. The term does not denote a particular age or sex. Thus, adult and newborn subjects, whether male or female, are intended to be covered. As used herein, “patient” or “subject” may be used interchangeably and can refer to a subject with a disease or disorder (e.g. cancer). The term patient or subject includes human and veterinary subjects. The term “subject in need thereof” as used herein refers to a subject diagnosed with or suspected of having cancer or an infectious disease as defined herein.

As used herein the terms “treatment”, “treat”, or “treating” refers to a method of reducing the effects of a disease or condition or symptom of the disease or condition. Thus, in the disclosed methods, treatment can refer to at least about 10%, at least about 20%, at least about 30%, at least about 40%, at least about 50%, at least about 60%, at least about 70%, at least about 80%, at least about 90%, or substantially complete reduction in the severity of an established disease or condition or symptom of the disease or condition, such as reduction in tumor volume, reduction in tumor burden, reduction in death. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms “prevent”, “preventing”, and “prevention” of a disease or disorder refers to an action, for example, administration of the chimeric polypeptide or nucleic acid sequence encoding the chimeric polypeptide, that occurs before or at about the same time a subject begins to show one or more symptoms of the disease or disorder, which inhibits or delays onset or exacerbation of one or more symptoms of the disease or disorder.

As used herein, references to “decreasing”, “reducing”, or “inhibiting” include a change of at least about 10%, of at least about 20%, of at least about 30%, of at least about 40%, of at least about 50%, of at least about 60%, of at least about 70%, of at least about 80%, of at least about 90% or greater as compared to a suitable control level. Such terms can include but do not necessarily include complete elimination of a function or property, such as agonist activity.

The term “sequence variant” refers to an amino acid sequence of a polypeptide that has substantially similar biological activity as a reference polypeptide but differs in amino acid sequence or to the nucleotide sequence of a nucleic acid that has substantially similar biological activity (e.g., encodes a protein with substantially similar activity) as a reference sequence but differs in nucleotide sequence. Typically the amino acid or nucleotide sequence of a “sequence variant” is highly similar (e.g. at least about 80% similar) to that of a reference sequence. Those of skill in the art readily understand how to determine the identity of two polypeptides or two nucleic acids. For example, the identity can be calculated after aligning the two sequences so that the identity is at its highest level over a defined number of nucleotides or amino acids. Optimal alignment of sequences for comparison may be conducted by the local identity algorithm of Smith and Waterman Adv. Appl. Math. 2:482 (1981), by the identity alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The term “conservative amino acid substitution” is a term of art that refers to the replacement of an amino acid in a polypeptide with another amino acid that has similar biochemical properties, such as size, charge and hydrophobicity as a reference amino acid. It is well-known that conservative amino acid replacements in the amino acid sequence of a polypeptide frequently do not significantly alter the overall structure or function of the polypeptide. Exemplary conservative substitutions are set forth in Table 4 below.

TABLE 4
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

The entire disclosures of all patent and non-patent publications cited herein are each incorporated herein by reference in their entireties for all purposes.

TABLE 5
Sequences

| SEQ ID | | |
|---|---|---|
| NO. | Name | Sequence |
| 1 | ACP457 | APTSSSTKKTQLQLEHLLLDLQMILNGINNYKNPKLTRMLTFKFYMPKKATEL |
| | (WW0621) | KHLQCLEEELKPLEEVLNLAQSKNFHLRPRDLISNINVIVLELKGSETTFMCEY |
| | | ADETATIVEFLNRWITFCQSIISTLTSGGPALFKSSFPPGSEVQLVESGGGLVQP |
| | | GNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSGGGGS |
| | | GGGGSGGGGSGGGGSGGGGSGGGGSSGGPALFKSSFPPGSEVQLVESGGGLV |
| | | QPGGSLRLSCAASGFTFSSYTLAWVRQAPGKGLEWVAAIDSSSYTYSPDTVRG |
| | | RFTISRDNAKNSLYLQMNSLRAEDTAVYYCARDSNWDALDYWGQGTTVTVS |
| | | SASTKGPSVFPLAPSSKSTSGGTAALGCLVKDYFPEPVTVSWNSGALTSGVHT |
| | | FPAVLQSSGLYSLSSVVTVPSSSLGTQTYICNVNHKPSNTKVDKRVEPKSC** |
| 2 | ACP378 | APTSSSTKKTQLQLEHLLLDLQMILNGINNYKNPKLTRMLTFKFYMPKKATEL |
| | (WW0520) | KHLQCLEEELKPLEEVLNLAQSKNFHLRPRDLISNINVIVLELKGSETTFMCEY |
| | | ADETATIVEFLNRWITFCQSIISTLTSGGPGPAGLYAQPGSEVQLVESGGGLVQP |
| | | GNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSGGGGS |
| | | GGGGSGGGGSGGGGSGGGGSGGGGSSGGPGPAGLYAQPGSEVQLVESGGGL |
| | | VQPGGSLRLSCAASGFTFSSYTLAWVRQAPGKGLEWVAAIDSSSYTYSPDTVR |
| | | GRFTISRDNAKNSLYLQMNSLRAEDTAVYYCARDSNWDALDYWGQGTTVTV |
| | | SSASTKGPSVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDKRVEPKSC** |
| 3 | WW0735 | APTSSSTKKTQLQLEHLLLDLQMILNGINNYKNPKLTRMLTFKFYMPKKATEL |
| | | KHLQCLEEELKPLEEVLNLAQSKNFHLRPRDLISNINVIVLELKGSETTFMCEY |
| | | ADETATIVEFLNRWITFCQSIISTLTSGGPGPAGLYAQPGSEVQLVESGGGLVQP |
| | | GNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSGGGGS |
| | | GGGGSGGGGSGGGGSGGGGSGGGGSGGGGSGGGGSGGGGSEVQLVESGGGL |
| | | VQPGGSLRLSCAASGFTFSSYTLAWVRQAPGKGLEWVAAIDSSSYTYSPDTVR |
| | | GRFTISRDNAKNSLYLQMNSLRAEDTAVYYCARDSNWDALDYWGQGTTVTV |
| | | SSASTKGPSVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDKRVEPKSC |
| 4 | WW0736 | APTSSSTKKTQLQLEHLLLDLQMILNGINNYKNPKLTRMLTFKFYMPKKATEL |
| | | KHLQCLEEELKPLEEVLNLAQSKNFHLRPRDLISNINVIVLELKGSETTFMCEY |
| | | ADETATIVEFLNRWITFCQSIISTLTSGGPALFKSSFPPGSEVQLVESGGGLVQP |
| | | GNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSGGGGS |
| | | GGGGSGGGGSGGGGSGGGGSGGGGSGGGGSGGGGSGGGGSEVQLVESGGGL |
| | | VQPGGSLRLSCAASGFTFSSYTLAWVRQAPGKGLEWVAAIDSSSYTYSPDTVR |
| | | GRFTISRDNAKNSLYLQMNSLRAEDTAVYYCARDSNWDALDYWGQGTTVTV |
| | | SSASTKGPSVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDKRVEPKSC |
| 5 | ACP381 | DIQMTQSPSSLSASVGDRVTITCKAREKLWSAVAWYQQKPGKAPKSLIYSASF |
| | (WW0523) | RYSGVPSRFSGSGSGTDFTLTISSLQPEDFATYYCQQYYTYPYTFGGGTKVEIK |
| | | RTVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGE |
| | | C |

### EXAMPLES

The following are examples of methods and compositions of the invention. It is understood that various other embodiments may be practiced, given the general description provided herein.

### Example 1: Inducible IL-2 and Anti-PD-1 Preclinical Models

In these examples the term “INDUKINE” is used to refer to inducible IL-2 prodrugs as disclosed in the detailed description.

Compound 1 has been evaluated in multiple mouse syngeneic tumor models, including MC38 colon carcinoma, B16F10 melanoma and CT26 colon carcinoma models and has demonstrated robust single agent anti-tumor activity and immune memory formation.

### Example 1A: MC38 Mouse Model

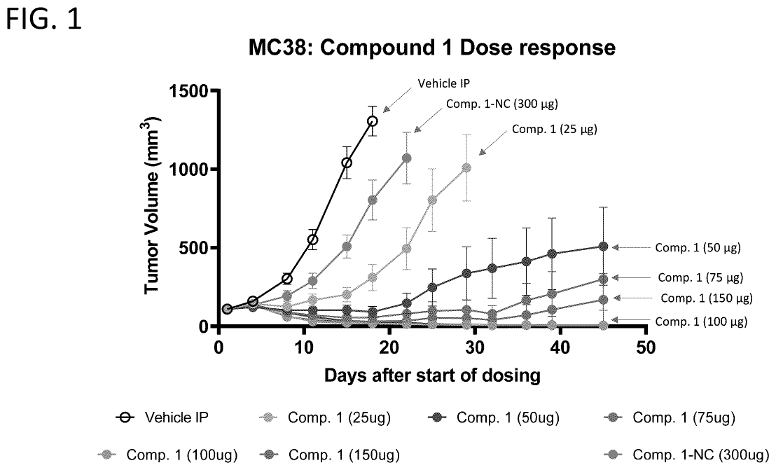

Mice were implanted with MC38 tumors and randomized into treatment groups when the tumors were between 100 to 150 mm³. Mice were then treated twice a week with titrated amounts of either a phosphate buffered solution (PBS) (acting as a vehicle), Compound 1, or a control IL-2 INDUKINE molecule engineered without the protease-cleavable linkers and, therefore, is not cleaved and does not release IL-2 in the tumor microenvironment (non-cleavable control). A total of 4 doses were administered. Treatment with the INDUKINE molecules were well tolerated by the mice, with no signs of body weight loss. As shown in FIG. 1, Compound 1 effectively treated the tumor in a dose dependent manner.

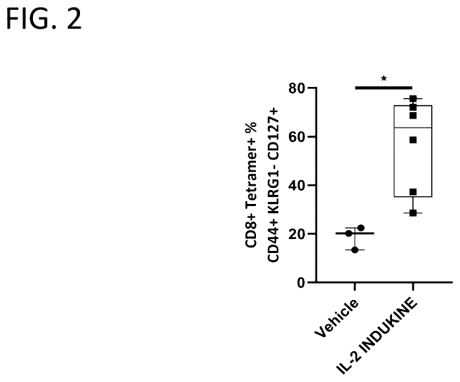

One hallmark of immunological rejection of a tumor is the development of protective memory against subsequent tumor rechallenge. To examine whether tumor rejection in animals treated with an IL-2 INDUKINE molecule resulted in immunological memory, spleens from animals that were treated with an IL-2 INDUKINE tool molecule (which has essentially the same biological properties as Compound 1) were examined for the presence of tumor-specific memory CD8+ T cells 40 days after the initial MC38 implantation. Tetramer staining was used to identify tumor specific CD8+ T cells, and those cells were examined for memory markers. As shown in FIG. 2, approximately 60% of the tetramer positive cells from the protected animals expressed the memory cell phenotype CD44+ KLRG1−CD127+ compared with only 20% of tetramer positive cells from control animals.

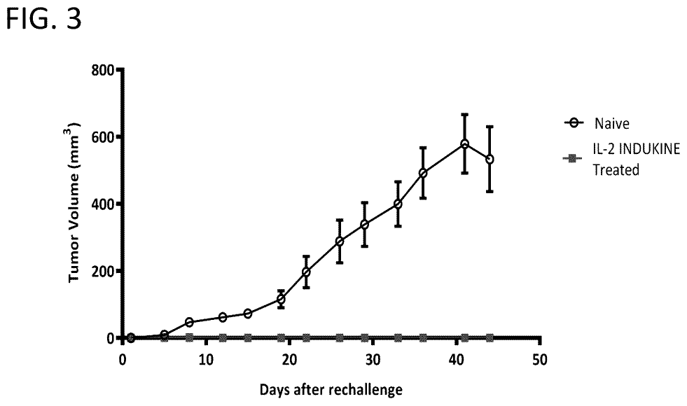

While the phenotype of these splenocytes suggests the generation of tumor specific memory, the ultimate test of a memory response is protection against rechallenge with the same tumor model. Therefore, mice that previously had complete MC38 tumor regressions following treatment with the IL-2 INDUKINE tool molecule were rechallenged by implanting more MC38 tumor cells 60 days after the initial implantation. No treatment was administered during the rechallenge. As shown in FIG. 3, unlike naive control animals who were also implanted with MC38 tumor cells, none of the animals previously treated with the IL-2 INDUKINE tool molecule developed tumors, suggesting that tumor rejection following prior IL-2 INDUKINE molecule treatment resulted in immunological memory.

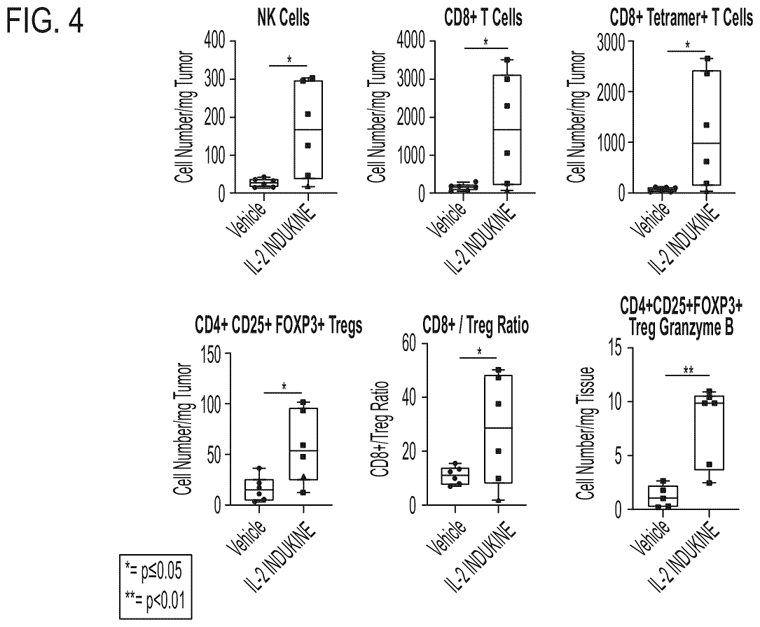

To better understand the mechanism by which the IL-2 INDUKINE molecule induced tumor regression, MC38 tumors from mice treated with either PBS or the IL-2 INDUKINE tool molecule were harvested 24 hours after their second dose in the first week to collect tumor infiltrating lymphocytes that were subsequently analyzed by flow cytometry. Five days after the initial dose, it was observed that treatment with the IL-2 INDUKINE tool molecule resulted in a large influx of immune cells, including NK cells, CD8+T effector cells and tumor specific tetramer+CD8+T effector cells. While there was an increase in the number of Tregs (defined as CD4+CD25+FOXP3+cells), the increase in CD8+ T cells far exceeded the increase in Tregs, resulting in a significant increase in the CD8+/Treg ratio (see FIG. 4). Additionally, treatment with the IL-2 INDUKINE tool molecule resulted in a subset of Tregs producing inflammatory cytokines such as Granzyme B, indicating that the Tregs that are expanded in tumors do not have a suppressive phenotype. Together, these data show that treatment with an IL-2 INDUKINE molecule increased immune cell tumor infiltration and activation in this model, thereby driving anti-tumor immunity.

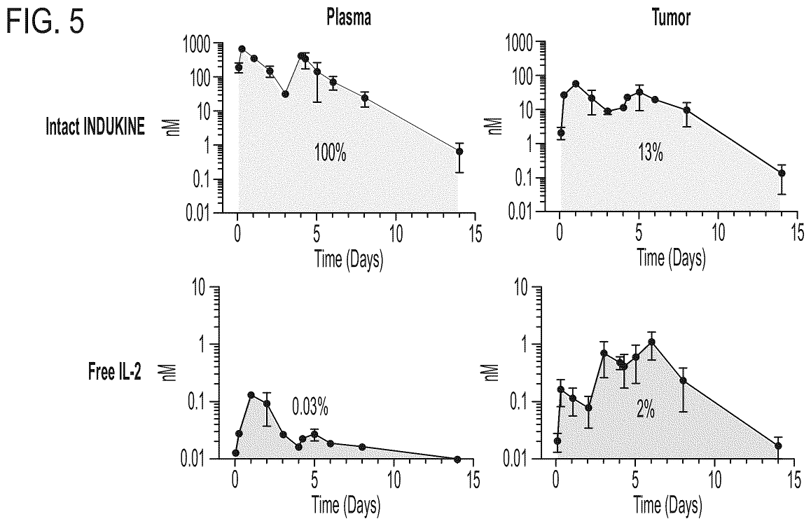

Conditionally activated, protease-cleavable linkers in the IL-2 INDUKINE molecule were observed to restrict the systemic activity of IL-2 while delivering IL-2 locally to the tumor. To test whether systemic dosing could result in localized delivery of IL-2 into the tumor, plasma and tumor samples were collected at various timepoints after dosing mice bearing MC38 tumors with the IL-2 INDUKINE tool molecule and analyzed for the presence of total, or intact, INDUKINE molecule as well as free IL-2 released due to activation of the INDUKINE molecule. As shown in FIG. 5, the exposure for the total INDUKINE molecule in plasma was approximately 8 fold higher than the exposure in the tumor, thus demonstrating favorable tumor tissue penetrance for the prodrug in this model. Low levels of free IL-2 were detected in the plasma, with 0.03% of the intact INDUKINE molecules in plasma processed to release free IL-2. In contrast, 2% of the total intact INDUKINE molecules in the tumor was processed to release IL-2. Furthermore, while the free IL-2 in the plasma reached maximum observed plasma concentration (Cₘₐₓ), at 24 hours post dosing, this exposure was transient. In contrast, the level of free IL-2 exposure in the tumor had a higher Cₘₐₓ and was sustained over time. This preferential activation of the INDUKINE molecule in the tumor results in an approximately 11-fold exposure of free IL-2 in tumors compared to the plasma. This suggests that tumor dependent processing drives the accumulation of IL-2 in the tumor following the systemic delivery of the IL-2 INDUKINE molecule.

### Example 1B. Non Human Primate Tolerability and PK

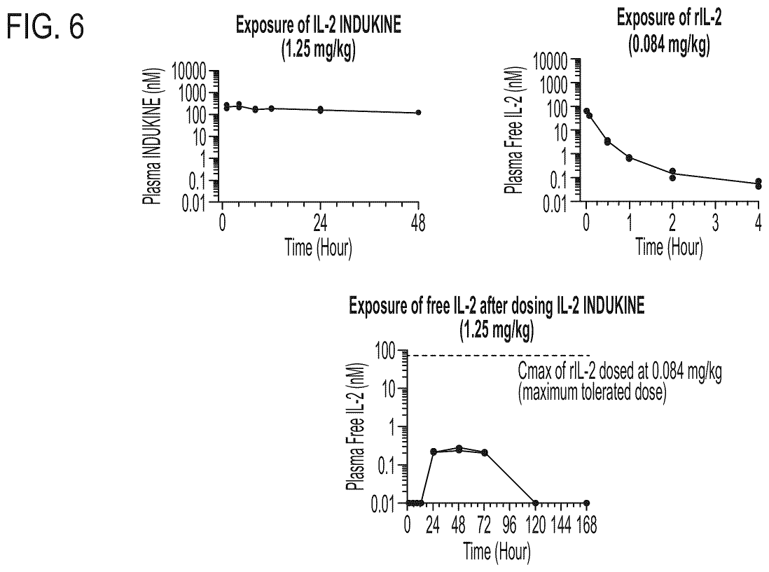

Compound 1 was administered to non-human primates (NHPs) in exploratory studies to determine the tolerability of Compound 1 and to measure the PK properties of both Compound 1 and IL-2 released from Compound 1. In the first study, animals were dosed with increasing amounts of Compound 1 from 0.05 mg/kg to 1.25 mg/kg once per week for 2 weeks. Plasma exposure of Compound 1 (measured as area under the concentration versus time curve (AUC) at a dose of 1.25 mg/kg was more than 500-fold higher than plasma exposure of rIL-2, at a dose of 0.084 mg/kg, its maximum tolerated dose (MTD), confirming that Compound 1 achieved high systemic exposure of IL 2, as shown in the top panels of FIG. 6 (left and right respectively). The mean half-life for Compound 1 after the first dose was approximately 57 hours, which was consistent across multiple dose levels. The plasma levels of free IL-2 released from Compound 1 were very low, with less than 0.01% of the plasma Compound 1 processed to release free IL-2, as shown in the bottom panel of FIG. 6. This confirms the stability of Compound 1 in circulation in the NHPs. Importantly, the Cₘₐₓ of circulating free IL-2 that could be measured following Compound 1 treatment of NHPs was significantly lower than the Cₘₐₓ of rIL-2 at its MTD. In a subsequent study, doses of up to 2 mg/kg of Compound 1 were well tolerated by the animals.

### Example 1C: Compound 1 in Combination with an Anti-PD-1 Antibody

Compound 1 was tested in a B16F10 syngeneic tumor model with or without the addition of an anti-PD1 antibody (RMP1-14). 1×10⁵ tumor cells were implanted in the flank of animals and tumor growth was monitored. Once tumors reached an average volume of 30-60 mm³, the animals were randomized and dosed as described in Table 6.

TABLE 6

| Group | N | Agent 1 | Dose | Agent 2 | Dose | | Schedule |
|---|---|---|---|---|---|---|---|
| 1 | 10 | Vehicle | N/A | N/A | 100 | μL | Biwk × 2 |
| 2 | 10 | Compound 1 | 100 μg/mouse | N/A | N/A | | Biwk × 2 |
| 3 | 10 | Compound 1 | 200 μg/mouse | N/A | N/A | | Biwk × 2 |
| 4 | 10 | N/A | N/A | Anti-PD-1 | 200 | μg/mouse | Biwk × 2 |
| 5 | 10 | Compound 1 | 100 μg/mouse | Anti-PD-1 | 200 | μg/mouse | Biwk × 2 |
| 6 | 10 | Compound 1 | 200 μg/mouse | Anti-PD-1 | 200 | μg/mouse | Biwk × 2 |

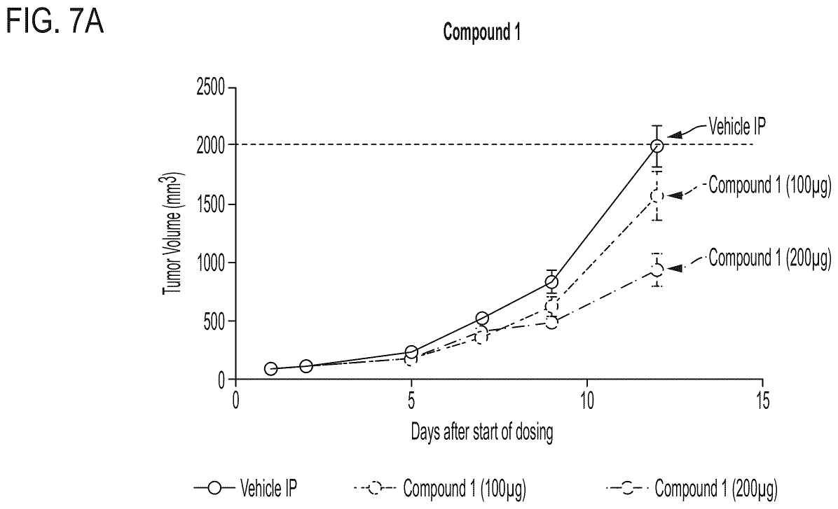

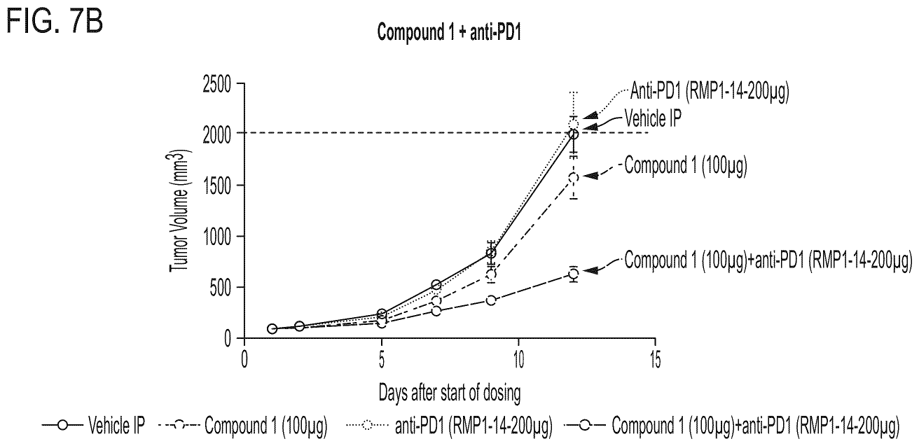

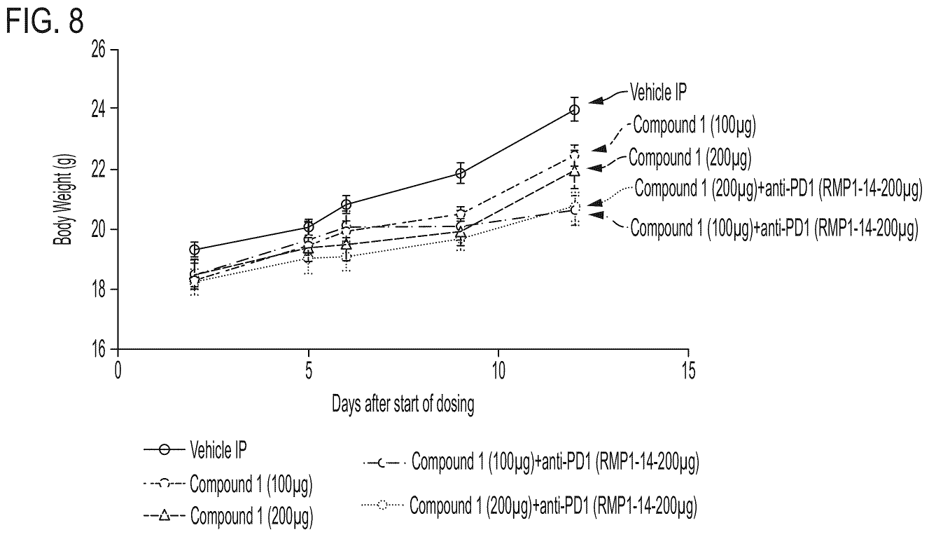

Tumor volumes and body weights were recorded three times per week with a gap of 2-3 days in between two measurements. Treatment with Compound 1 showed dose dependent efficacy as a monotherapy. Monotherapy with anti-PD1 in this model had no efficacy and tumor volumes in anti-PD1 treated mice were similar to those in mice treated with vehicle only. But the combination therapy using Compound 1 and an anti-PD1 antibody synergistically improved tumor control, and was more effective than either Compound 1 or an ani-PD-1 antibody monotherapy. The results are shown in FIGS. 7A, 7B, and 8.

### Example 2: A Multicenter Phase I/b Dose Escalation Study of IL-2 Prodrug as Monotherapy and in Combination with Pembrolizumab in Patients with Selected Advanced or Metastatic

### Solid Tumors

TABLE 7
List of Abbreviations and Definition of Terms

| Abbreviation | Definition |
|---|---|
| ε | Fraction of error |
| ADA | Anti-drug antibody |
| AESI | Adverse event of special interest |
| ALT | Alanine aminotransferase |
| AST | Aspartate aminotransferase |
| AUC | Area under the concentration versus time curve |
| AUC_{0-inf} | Area under the concentration versus time curve from time 0 to infinity |
| AUC₀₋ₜ | Area under the concentration versus time curve from time 0 to t |
| AV | Atrioventricular |
| BRAF | V-raf murine sarcoma viral oncogene homolog B1 |
| CD | Cluster of differentiation |
| CFR | Code of Federal Regulations |
| CL | Clearance |
| CLS | Capillary leak syndrome |
| Cₘₐₓ | Maximum observed plasma concentration |
| Cₘᵢₙ | Minimum observed plasma concentration |
| CNS | Central nervous system |
| CPI | Checkpoint inhibitor |
| CR | Complete response |
| CRA | Clinical Research Associate |
| CRP | C-reactive protein |
| CRS | Cytokine release syndrome |
| CT | Computed tomography |
| CTLA-4 | Cytotoxic T-lymphocyte-associated protein 4 |
| CYP | Cytochrome P450 |
| D | De-escalate to the next lower dose |
| DEC | Dose Escalation Committee |
| DLT | Dose-limiting toxicity |
| DRF | Dose range finding |
| DNA | Deoxyribonucleic acid |
| DOR | Duration of response |
| DU | De-escalate to a lower dose and never test this dose again |
| E | Escalate to the next higher dose |
| ECG | Electrocardiogram |
| ECOG | Eastern Cooperative Oncology Group |
| eCRF | Electronic Case Report Form |
| EDC | Electronic Data Capture |
| EIU | Exposure in Utero |
| EOT | End of Treatment |
| FDA | Food and Drug Administration |
| FIH | First-in-human |
| FOXP3 | Forkhead box P3 |
| GCP | Good Clinical Practice |
| HCV | Hepatitis C virus |
| HD | High-dose |
| HIV | Human Immunodeficiency virus |
| IB | Investigator's Brochure |
| ICF | Informed consent form |
| ICH | International Council for Harmonisation |
| iCPD | Immune-confirmed progressive disease |
| iCR | Immune-complete response |
| iDOR | Immune-duration of response |
| IFN | Interferon |
| Ig | Immunoglobulin |
| IgG4 | Immunoglobulin G4 |
| IgV | Ig-variable |
| IL | Interleukin |
| IL-2R | Interleukin-2 receptor |
| iORR | Immune-overall response rate |
| iPFS | Immune-progression-free survival |
| iPR | Immune-partial response |
| irAE | Immune-related adverse events |
| IRB | Institutional Review Board |
| iRECIST | Immune-Response Evaluation Criteria in Solid Tumors |
| iUPD | Immune-unconfirmed progressive disease |
| IV | Intravenous(ly) |
| KLRG1 | Killer cell lectin-like receptor subfamily G member 1 |
| L | Ligand |
| MAPK | Mitogen-activated protein kinase |
| MedDRA | Medical Dictionary for Regulatory Activities |
| MEK | Mitogen-activated protein kinase kinase |
| MR | Magnetic resonance |
| mRCC | Metastatic renal cell carcinoma |
| mRNA | Messenger ribonucleic acid |
| MTD | Maximum tolerated dose |
| mTPI | Modified Toxicity Probability Interval |
| NCI CTCAE | National Cancer Institute Common Terminology Criteria for Adverse Events |
| NHP | Non-human primate |
| NK | Natural killer |
| ORR | Overall response rate |
| PBS | Phosphate buffered solution |
| PD | Pharmacodynamic(s) |
| PD-1 | Programmed cell death 1 |
| PD-L1 | Programmed cell death ligand 1 |
| PD-L2 | Programmed cell death ligand 2 |
| PFS | Progression-free survival |
| PK | Pharmacokinetic(s) |
| PR | Partial response |
| Q2W | Every 2 weeks |
| Q3W | Every 3 weeks |
| Q6W | Every 6 weeks |
| qPCR | Quantitative polymerase chain reaction |
| QTcF | QT interval corrected for heart rate using Fridericia's formula |
| RDE | Recommended dose for expansion |
| RECIST | Response Evaluation Criteria in Solid Tumors |
| rIL-2 | Recombinant interleukin-2 |
| RNA | Ribonucleic acid |
| S | Stay at the current dose |
| SAE | Serious adverse events |
| SHP | Src homology 2 domain-containing protein tyrosine phosphatase |
| SUSAR | Suspected Unexpected Serious Adverse Reaction |
| t_{1/2} | Terminal-elimination half-life |
| TEAE | Treatment-emergent adverse event |
| Tₘₐₓ | Time to maximum observed plasma concentration |
| TME | Tumor microenvironment |
| Treg | Regulatory T cell |
| T-VEC | Talimogene laherparepvec |
| ULN | Upper limit of normal |
| USA | United States of America |
| V_{d} | Volume of distribution |
| VEGFI | Vascular endothelial growth factor inhibitor |
| VLS | Vascular Leak Syndrome |
| WT | Wild type |

### INTRODUCTION AND BACKGROUND INFORMATION

### Interleukin-2

Interleukin (IL)-2 is a pro-inflammatory cytokine which can drive the immune-mediated killing of cancer cells through the proliferation and activation of T cells and natural killer (NK) cells and inducing the differentiation of cluster of differentiation (CD)8 cells into effector and memory cells. The IL-2 receptor (IL-2R) is composed of 3 subunits named IL 2Rα (CD25), IL-2Rβ (CD122), and IL-2Rγ(CD132). Binding to monomeric IL-2Rα does not induce signaling, while binding to the medium affinity dimeric receptor comprised of a complex of the β and γ subunits will induce signaling. The trimeric receptor composed of all 3 subunits is a high affinity receptor for IL-2, with binding affinity approximately 100-fold higher than the medium affinity receptor. Binding to the medium or high affinity IL-2R activates the Janus kinase/signal transducer and activator of transcription (STAT), mitogen-activated protein kinase (MAPK), and phosphoinositide 3-kinase signaling pathways in target immune cells resulting in immune cell activation and proliferation.

The medium affinity IL-2Rβ/γ is expressed on NK cells, monocytes, macrophages, and resting CD4+ and CD8+ T cells, while the high affinity IL-2Rα/β/γ is transiently induced on activated T and NK cells and is constitutively expressed on CD4+FoxP3+regulatory T cells (Tregs). Basal levels of IL-2 bind predominantly to high affinity IL-2Rα/β/γ on Tregs to maintain immune homeostasis. IL-2 production during an immune response results in levels of IL-2 which can activate both the medium and high affinity receptors, resulting in the activation and proliferation of effector lymphocyte populations.

Numerous preclinical studies have demonstrated that administration of IL-2 can be effective in eradicating tumors in mouse models. This concept has also been clinically validated with the approval in 1992 of recombinant IL-2 (rIL-2) therapy (aldesleukin) for renal cell carcinoma and in 1998 for metastatic melanoma. Aldesleukin has demonstrated complete cancer regression in about 10% of patients treated for metastatic melanoma and renal cancer. Unfortunately, rIL-2 has poor pharmacokinetic (PK) properties and dose limiting systemic toxicities due to binding to its high and medium affinity receptors in the periphery. High dose IL-2 administration results in severe hypotension and vascular leak syndrome (VLS), which has relegated its use to specialized care centers and limited its dosing to reach efficacious levels. These side effects limit the number of patients who can tolerate the recommended therapeutic regimen and, consequently, achieve the full clinical benefit from IL-2 therapy. It has been postulated that another contributing factor limiting the clinical benefit of IL-2 is that by binding to the high-affinity IL-2Rα/β/γ, it induces the expansion of immunosuppressive Tregs, which can counteract anti-tumor immune responses.

### IL-2 Prodrug

The IL-2 prodrug is a systemically delivered, conditionally activated, form of interleukin 2 designed to minimize the severe toxicities observed with rIL-2 therapy and maximize clinical benefit when administered as monotherapy or in combination with immune checkpoint inhibitors in advanced or metastatic tumors.

The IL-2 prodrug is engineered with a native IL-2 molecule attached via protease-cleavable linkers to both an IL-2Rβ/γ blockade element to eliminate binding of the IL-2 to IL-2Rβ/γ-expressing normal tissues in the periphery and a half-life extension domain. The prodrug is conditionally activated in the tumor microenvironment through protease cleavage to release the fully active, native IL-2 cytokine within the tumor to stimulate a potent anti-tumor immune response.

Several companies are also developing IL-2 therapies designed to address the limitations of rIL-2 by engineering molecules that bind only to the medium affinity receptor IL-2Rβ/γ and avoid binding to the high affinity receptor IL-2Rα/β/γ, so called “non-alpha” molecules, in the hope of alleviating toxicities and reducing activation of Tregs. However, many of these molecules activate IL-2Rβ/γ receptors in the periphery (due to lack of an IL-2Rβ/γ blockade element) and are also attenuated in inducing newly primed T cell proliferation in the tumor microenvironment (TME) due to their reduced IL- 2Rα binding, which may limit their ability to reach efficacious exposures. Binding to the high affinity receptor IL- 2Rα/β/γ may be necessary for optimal anti-tumor activity as recent published work shows. First, analysis of T cell clonal content and activation state in patients with basal cell carcinoma after positive response to anti-programmed cell death 1 (PD-1) therapy showed that those tumors go through a process of clonal replacement where most of the activated/exhausted T cells found after treatment are newly activated clones that express the high affinity receptor IL-2Rα/β/γ during expansion. These cells will benefit from the presence of exogenous fully active IL-2 during this process. Second, signaling through the high affinity receptor IL-2Rα/β/γ plays a critical role directing the generation of effective memory formation and secondary responses.

The specific design features of the IL-2 prodrug will address these challenges by inhibiting the interaction of IL-2 with the IL-2Rβ/γ in the periphery to minimize systemic toxicity while delivering a native IL-2 into the TME with IL-2Rα/β/γ binding to realize the full pharmacology of IL-2 in driving anti-tumor immune responses. In certain embodiments, the IL-2 prodrug used in this Example 2 is Compound 1. In certain embodiments, the IL-2 prodrug used in this Example 2 is Compound 2. In certain embodiments, the IL-2 prodrug used in this Example 2 is Compound 3. In certain embodiments, the IL-2 prodrug used in this Example 2 is Compound 4.

### Pembrolizumab

Pembrolizumab is a potent humanized immunoglobulin (Ig) G4 (IgG4) monoclonal antibody with high specificity of binding to the programmed cell death 1 (PD-1) receptor, thus inhibiting its interaction with programmed cell death ligand 1 (PD-L1) and programmed cell death ligand 2 (PD-L2). Based on preclinical in vitro data, pembrolizumab has high affinity and potent receptor blocking activity for PD-1. Pembrolizumab has an acceptable preclinical safety profile and is in clinical development as an intravenous (IV) immunotherapy for advanced malignancies. KEYTRUDA® (pembrolizumab) is indicated for the treatment of patients across a number of indications.

Refer to the approved labeling for detailed background information on pembrolizumab.

### Pharmaceutical and Therapeutic Background

The importance of intact immune surveillance function in controlling outgrowth of neoplastic transformations has been known for decades. Accumulating evidence shows a correlation between tumor-infiltrating lymphocytes in cancer tissue and favorable prognosis in various malignancies. In particular, the presence of CD8+ T-cells and the ratio of CD8+ effector T cells/FoxP3+ Tregs correlates with improved prognosis and long-term survival in solid malignancies, such as ovarian, colorectal, and pancreatic cancer, hepatocellular carcinoma, malignant melanoma, and renal cell carcinoma. Tumor-infiltrating lymphocytes can be expanded ex vivo and reinfused, inducing durable objective tumor responses in cancers such as melanoma.

The PD-1 receptor-ligand interaction is a major pathway hijacked by tumors to suppress immune control. The normal function of PD-1, expressed on the cell surface of activated T cells under healthy conditions, is to down-modulate unwanted or excessive immune responses, including autoimmune reactions. PD-1 (encoded by the gene PDCD1) is an Ig superfamily member related to CD28 and cytotoxic T-lymphocyte-associated protein 4 (CTLA-4) that has been shown to negatively regulate antigen receptor signaling upon engagement of its ligands (PD-L1 and/or PD L2).

The structure of murine PD-1 has been resolved. PD-1 and its family members are type 1 transmembrane glycoproteins containing an Ig-variable (IgV)-type domain responsible for ligand binding and a cytoplasmic tail responsible for the binding of signaling molecules. The cytoplasmic tail of PD-1 contains 2 tyrosine-based signaling motifs, an immunoreceptor tyrosine based inhibition motif, and an immunoreceptor tyrosine-based switch motif. Following T cell stimulation, PD-1 recruits the tyrosine phosphatases, src homology 2 domain-containing protein tyrosine phosphatase (SHP)-1 and SHP-2, to the immunoreceptor tyrosine-based switch motif within its cytoplasmic tail, leading to the dephosphorylation of effector molecules such as CD3ζ, protein kinase Cθ, and zeta-chain-associated protein kinase 70, which are involved in the CD3 T cell signaling cascade. The mechanism by which PD-1 down-modulates T cell responses is similar to, but distinct from, that of CTLA-4 because both molecules regulate an overlapping set of signaling proteins. As a consequence, the PD-1/PD-L1 pathway is an attractive target for therapeutic intervention in combination with a pro-inflammatory T cell directed mechanism, such as that generated by the IL-2 prodrugs disclosed herein.

### Study Rationale

Immunotherapy, which includes proinflammatory cytokines like rIL-2, has become a well-established treatment modality for multiple cancer indications. rIL-2 therapy demonstrated dramatic clinical activity with durable complete responses in metastatic renal cell carcinoma (mRCC) and cutaneous malignant melanoma, leading to its Food and Drug Administration (FDA) approval for those indications in 1992 and 1998, respectively. However, administration of high dose IL-2 therapy has been limited due to its short half-life, poor pharmaceutical properties, and severe toxicities requiring it to be administered to patients under monitored conditions in the Intensive Care Unit. Therefore, it has been replaced over time by other more tolerable therapies, including checkpoint inhibitors such as PD-L1 antagonists and targeted therapies. Data from the ProleukinR Observational Study to Evaluate the Treatment Patterns and Clinical Response in Malignancy registry, a national observational database established to document and study the current treatment outcomes with high-dose (HD) IL-2, have shown that overall response rates and survival benefit of HD IL-2 in metastatic melanoma and mRCC sequenced after or in combination with Standard of Care therapies, including prior checkpoint inhibitor therapy, are consistent with previously reported data showing durable, long-term responses.

Despite advances in cancer care and the demonstration that numerous tumor types beyond malignant melanoma and mRCC are sensitive to checkpoint blockade, there remains an unmet medical need for a next-generation, full-potency IL-2 that can be safely administered. The IL-2 prodrug is a systemically delivered, conditionally activated IL-2 INDUKINE molecule that is being developed to minimize the severe toxicities observed with rIL-2 therapy and maximize clinical benefit when administered as monotherapy or in combination with an immune checkpoint inhibitor (CPI) in immunosensitive advanced or metastatic solid tumors.

The IL-2 prodrug as a monotherapy or in combination with CPI in tumor types for which CPI therapy has demonstrated activity has the potential to safely deliver the full biological potency of IL-2 and result in a powerful anti-tumor immune response.

Data from cynomolgus monkeys have demonstrated a wide therapeutic index with the safe delivery of the IL-2 prodrug at doses associated with efficacious exposures in mouse tumor models. The combination of the IL-2 prodrug and anti-PD-1 in anti-PD-1 resistant models has shown synergistic anti-tumor activity.

This is a Phase 1/lb dose escalation and expansion study to determine the safety, tolerability, PK, and preliminary anti-tumor activity of the systemic administration of the IL-2 prodrug alone or in combination with pembrolizumab in selected advanced or metastatic solid tumors and to identify the doses and schedule appropriate for further study.

### Rationale for IL-2 Prodrug Starting Dose

The starting dose for the IL-2 prodrug will be selected based on IND enabling study data or GLP toxicology data and will be delivered systemically every 2 weeks (Q2W). The current proposed doses of the IL-2 prodrug are: 1, 3, 10, 30, 60, 120, and 240 mg. The predicted Cₘₐₓ and AUC for the 3 mg dose are expected to have a 65-fold and 31-fold exposure margin, respectively, when compared with the top dose administered in the DRF NHP toxicology study. The 3 mg dose (0.542 nmol/kg for a 70 kg patient) is also lower than the recommended dose of rIL-2 (2.387 nmol/kg) given every 8 hours for 5 consecutive days, and, for all dose levels of the IL-2 prodrug, systemic exposure to free IL-2 is expected to be below the maximum tolerated exposure for rIL 2 given as a continuous IV infusion. Confirmation of the IL-2 prodrug first in human (FIH) starting dose and rationale will be finalized following the conduct of the GLP toxicology study.

### Rationale for Pembrolizumab Dose

The planned dose of pembrolizumab for this study is 400 mg every 6 weeks (Q6W).

A 400 mg Q6W dosing regimen of pembrolizumab is expected to have a similar benefit-risk profile as 200 mg every 3 weeks (Q3W), in all treatment settings in which 200 mg Q3W pembrolizumab is currently appropriate. Specifically, the dosing regimen of 400 mg Q6W for pembrolizumab is considered adequate based on modeling and simulation analyses, given the following rationale:
PK simulations demonstrating that in terms of pembrolizumab exposures—
   Average concentration over the dosing interval (or AUC) at 400 mg Q6W is similar to that at the approved 200 mg Q3W dose, thus bridging efficacy between dosing regimens;
   Trough concentrations (Cₘᵢₙ) at 400 mg Q6W are generally within the range of those achieved with 2 mg/kg or 200 mg Q3W in the majority (>99%) of patients;
   Peak concentrations (Cₘₐₓ) at 400 mg Q6W are well below the Cₘₐₓ for the highest clinically tested dose of 10 mg/kg Q2W, supporting that the safety profile for 400 mg Q6W should be comparable to the established safety profile of pembrolizumab; and
   Exposure-response for pembrolizumab has been demonstrated to be flat across indications, and overall survival predictions in melanoma and non-small cell lung cancer demonstrate that efficacy at 400 mg Q6W is expected to be similar to that at 200 mg or 2 mg/kg Q3W, given the similar exposures; thus 400 mg Q6W is expected to be efficacious across indications.

### Risk/Benefit

Patients enrolled in this study will be those with metastatic malignancies who have limited treatment options. Appropriate eligibility criteria and specific dose-limiting toxicity (DLT) definitions, as well as specific monitoring guidelines and dose modification and individual patient stopping rules are included in this protocol.

### STUDY OBJECTIVES

### Primary Objectives

The primary objectives of the Dose Escalation Phase of this study are the following:
To determine the MTD and/or recommended dose for expansion (RDE) and evaluate the safety and tolerability of the IL-2 prodrug; and
To determine the MTD and/or RDE and evaluate the safety and tolerability of the IL-2 prodrug in combination with pembrolizumab.

The primary objectives of the Expansion Phase of this study are the following:
To further characterize the safety of the IL-2 prodrug when administered as monotherapy and in combination with pembrolizumab; and
To evaluate the anti-tumor activity of the IL-2 prodrug (as monotherapy and in combination with pembrolizumab) in advanced or metastatic renal clear cell carcinoma and advanced or metastatic cutaneous malignant melanoma, as measured by overall response rate (ORR) (complete response [CR]+partial response [PR]) by Response Evaluation Criteria in Solid Tumors (RECIST) 1.1 and immune ORR (iORR) (iORR=immune-CR+immune-PR) by immune-RECIST (iRECIST).

### Secondary Objectives

The secondary objectives of this study are the following:
To characterize the PK profile of the IL-2 prodrug (parent compound and free IL-2);
To evaluate the anti-tumor activity of the IL-2 prodrug, as measured by ORR(CR+PR), DOR, and PFS by RECIST 1.1 and iRECIST;
To evaluate the anti-tumor activity of the IL-2 prodrug in combination with pembrolizumab, as measured by ORR(CR+PR), DOR, and PFS by RECIST 1.1 and iORR (iORR=immune complete response [iCR]+immune-partial response [iPR]), iDOR, and iPFS by iRECIST;
To evaluate the changes in certain immunological biomarkers in blood and in baseline and post-treatment tumor biopsies in response to the IL-2 prodrug as monotherapy or in combination with pembrolizumab;
To evaluate the anti-tumor activity of the IL-2 prodrug (as monotherapy and in combination with pembrolizumab) in advanced or metastatic renal cell carcinoma and advanced or metastatic cutaneous malignant melanoma, as measured by duration of response (DOR) and progression-free survival (PFS) by RECIST 1.1 and immune-RECIST (iRECIST); and
To evaluate immunogenicity of the IL-2 prodrug, including the potential to generate an anti-drug antibody (ADA) response.

### Exploratory Objectives

The exploratory objectives of this study are the following:
To evaluate pharmacodynamic (PD) activities of the IL-2 prodrug (alone and in combination with pembrolizumab);
To investigate immunological biomarkers in peripheral blood and tumor that may correlate with the treatment outcome of the IL-2 prodrug as monotherapy or in combination with pembrolizumab; and
To assess tumor biopsies for potential biomarkers of target engagement and immune pathway activation.

### Study Description

This is a FIH, open-label, multicenter study to determine the safety profile and MTD and/or RDE of the IL-2 prodrug alone or in combination with pembrolizumab.

The study will enroll patients in the monotherapy and combination arms of the Dose Escalation Phase with relapsed/refractory locally advanced or metastatic immunotherapy-sensitive solid tumors, defined as specific indications for which CPIs are approved, who have progressed on or are intolerant to standard therapy, or for whom no standard therapy with proven benefit exists. Patients will have received prior CPI therapy, including prior anti-PD-1 or -(L)1 inhibitors alone or in combination with other agents. Patients in the combination arm should have discontinued that therapy due to either disease progression or reasons other than toxicity. Patients in all phases of the study may not have received prior IL 2 directed therapy.

Patients will be enrolled in the monotherapy and combination therapy arms of the Dose Expansion Phase who have advanced or metastatic renal clear cell carcinoma or advanced or metastatic cutaneous malignant melanoma. For monotherapy dose expansion, patients with mRCC may have received up to 3 prior lines of therapy and must have received prior anti-angiogenic therapy (VEGFi) and a CPI. Patients with v-raf murine sarcoma viral oncogene homolog B1 (BRAF) wild-type cutaneous malignant melanoma must have received anti-PD-(L)1 inhibitor therapy alone or in combination with a CTLA-4 inhibitor or other therapy. Patients with BRAF mutant melanoma may have received 2 prior lines of therapy, which included a CPI and a BRAF inhibitor with or without a MEK inhibitor.

For the mRCC patients in the combination part of the Dose Expansion Phase specifically, patients must have had no more than 2 prior lines of therapy, only 1 of which included an anti-PD-(L)1 inhibitor therapy alone or in combination. Cutaneous malignant melanoma patients enrolled in the combination part of the Dose Expansion Phase may have received 0-2 prior lines of therapy depending upon BRAF mutation status. Patients in the combination therapy arms of the dose expansion phase should not have discontinued prior CPI therapy for immune-related toxicities.

The assignment of a patient to a particular dose cohort during dose escalation and to a particular arm during dose expansion will be coordinated by Sponsor. When monotherapy and combination therapy arms are open in parallel, eligible patients will be enrolled sequentially into available slots.

The overall sample size for this study is approximately 150 patients between the Dose Escalation Phase and Dose Expansion Phase but will depend on the observed DLT profiles of the IL-2 prodrug monotherapy and the IL-2 prodrug in combination with pembrolizumab.

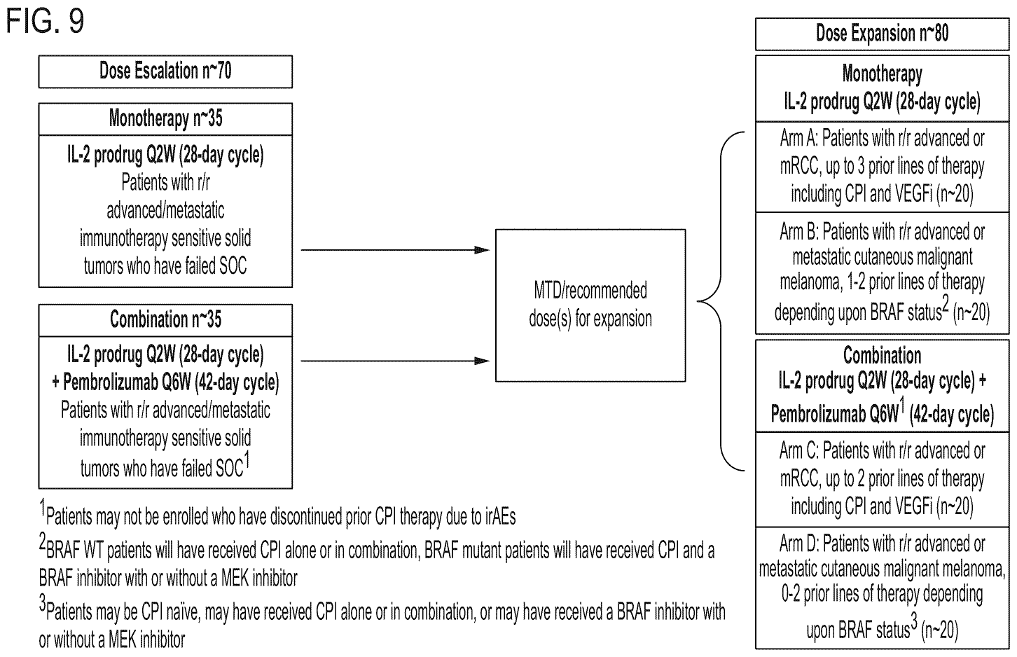

An overall study design schematic is presented in FIG. 9.

### Dose Escalation Phase

Dose escalation will utilize a modified Toxicity Probability Interval (mTPI)-2 design. The starting dose of the IL-2 prodrug may be a 1 mg or 3 mg flat dose administered IV every 2 weeks (Q2W), and the proposed doses, if supported by mTPI-2, may be 1, 3, 10, 30, 60, 120, and 240 mg IV Q2W. Intermediate doses may be evaluated. Modifications in the dosing regimen to Q3W may be made based on the PK and safety profiles observed and will be implemented by amendment. A stagger of at least 2 days is required between dosing of the first and second patients at each new untested dose level, and a stagger of 1 day is required between dosing of the second and third patients.

Dose confirmation using an mTPI-2 design

An mTPI-2 design with a target DLT rate of approximately 30% and an acceptable DLT range of 25% to 35% (ε1=ε2=0.05) will be applied for determining the MTD and/or RDE for the IL-2 prodrug.

The current proposed doses of the IL-2 prodrug are: 1, 3, 10, 30, 60, 120, and 240 mg. The predicted Cₘₐₓ. and AUC for the 3 mg dose are expected to have a 65 fold and 31 fold exposure margin, respectively, when compared with the top dose administered in the GLP NHP toxicology study (FIH starting dose will be finalized after completion of GLP toxicology study). The 3 mg dose (0.542 nmol/kg for a 70 kg patient) is also lower than the recommended dose of rIL 2 (2.387 nmol/kg) given every 8 hours for 5 consecutive days and for all dose levels of the IL-2 prodrug, and systemic exposure to free IL-2 is expected to be below the maximum tolerated exposure for rIL-2 given as a continuous IV infusion. Both total parent drug (the IL-2 prodrug) and free IL-2 measurements will be performed and may be considered when selecting the dose for the next cohort based on the PK and the safety profiles observed.

Escalation or de-escalation decisions will be based on the occurrence of DLTs and adverse events, particularly severe immune-related toxicities, at a given dose for the first 28-day period (Cycle 1) and will be made by the Dose Escalation Committee (DEC).

During the continuous safety assessment phase, a minimum of 3 patients are required at each dose. Depending on accrual rate and occurrence of DLTs, 3, 4, 5, or 6 patients may be enrolled at each new dose until the last of those patients completes the 28-day DLT assessment period. For example, if 1 out of the first 3 patients at a given dose level develops a DLT, no more than an additional 3 patients should be enrolled at this dose level until additional DLT data are available since this dose will be de-escalated if 2 of the additional patients experience a DLT (i.e., 3 out of 5 patients). If 2 out of the first 3 patients at a given dose level develop a DLT, the dose will be de-escalated to the next lower level. If 3 out of the first 3 patients at a given dose level develop a DLT, this dose will be considered unacceptably toxic (i.e., the dose will be de-escalated and never re-escalated to that dose again). If a lower dose is used, and 0 out of the next 3 patients at a given dose level develops a DLT, then the dose can be re-escalated back to the next level. The same principle will be applied whether 3, 4, 5, or 6 patients are enrolled in the same dose cohort.

Based on the mTPI-2 design, the number of patients who are enrolled at a dose, but are not yet fully evaluable for DLT assessment, may not exceed the number of remaining patients who are at risk of developing a DLT before the dose would be considered unacceptably toxic. In total, 3 to 12 patients may be enrolled at a given dose level for the continuous safety assessment phase.

Dose escalation and confirmation of the RDE will generally end after up to 12 patients have been treated at any of the selected doses found to be acceptable. The MTD is defined as the dose level that can be given such that the estimated DLT probability is closest to approximately 30%. Estimation of the MTD will be based on the estimation of the incidence rate of DLTs. The totality of the data will be considered before a dose is selected to carry forward to further enrollment and the escalation schedule may be adjusted based on PK and safety data emerging throughout the study to determine the RDE.

Note that while 30% was the target toxicity rate used to generate the guidelines, the observed rates of patients with DLTs at the MTD may be slightly above or below 30%.

Dose Escalation Phase with Pembrolizumab

Dose escalation for the first combination cohort will begin for the IL-2 prodrug in combination with pembrolizumab following completion of the third monotherapy dose cohort (i.e., in parallel to the fourth monotherapy dose cohort). The dose escalation for the IL-2 prodrug in combination with pembrolizumab cohorts will follow an mTPI 2 design using the same rules as described above for monotherapy with the starting dose level for the IL-2 prodrug at the dose level that is 1 dose level below the highest dose determined to be safe in the monotherapy dose escalation at that time. For each new dose level, the the IL-2 prodrug dose level to be tested in combination with pembrolizumab must have been shown to be safe and tolerable as monotherapy. The pembrolizumab dose will be administered at the approved dose of 400 mg Q6W and will not be escalated or de-escalated.

### Dose-Limiting Toxicity Criteria

Toxicity will be evaluated according to the National Cancer Institute-Common Terminology Criteria for Adverse Events (NCI CTCAE) version 5.0. The DLT observation period for both monotherapy the IL-2 prodrug and pembrolizumab combination dose escalation is 28 days. A DLT is defined as an adverse event that is at least possibly related to study drug and not reasonably attributed to the patient's underlying disease, other medical conditions, or concomitant medications or procedures.

study Stopping Rules

Beyond Cycle 1 in the escalation part and during the expansion part, the study will use the DLT criteria, and if observed toxicities meeting these DLT criteria in ≥33% of subjects at any point in time, the DEC will be convened to review the available safety data and determine appropriate subsequent steps (e.g. refinement of dose, modification of study assessments, study closure, etc.). Enrollment may be paused while such a review is undertaken. Any death that is considered possibly related to the IL-2 prodrug alone or in combination with pembrolizumab, occurring within 28 days of receiving the first dose(s) of study treatment, will result in a study enrollment pause and cessation of dosing at the current dose level, to allow for an expedited ad hoc evaluation by the DEC prior to further enrollment in the study.

### Dose Expansion Phase

Once the respective MTD(s) and/or RDE(s) are determined for the IL-2 prodrug as monotherapy or in combination with pembrolizumab in the Dose Escalation Phase, patients will be enrolled into the respective expansion arms for the tumor types specified below, 2 monotherapy expansion arms and 2 combination expansion arms, with up to 20 patients enrolled in each. Expansion arms for monotherapy and combination therapy may open and begin enrollment independently. Based on evolving safety and efficacy data, additional patients may be enrolled by amendment. The 4 expansion cohorts will include the following: 1. Arm A: the IL-2 prodrug as a monotherapy at the RDE in advanced or mRCC; 2. Arm B: the IL-2 prodrug as a monotherapy at the RDE in advanced or metastatic cutaneous malignant melanoma; 3. Arm C: the IL-2 prodrug at the RDE in combination with pembrolizumab in advanced or mRCC; and 4. Arm D: the IL-2 prodrug at the RDE in combination with pembrolizumab in advanced or metastatic cutaneous malignant melanoma.

### Selection of Study Population

The study will enroll patients in the monotherapy and combination arms of the Dose Escalation Phase with relapsed/refractory locally advanced or metastatic immunotherapy-sensitive solid tumors, defined as specific indications for which CPIs are approved, who have progressed on or are intolerant to standard therapy, or for whom no standard therapy with proven benefit exists. Patients will have received prior CPI therapy, including prior anti-programmed death 1 (PD-1) or-(L)1 inhibitors alone or in combination with other agents. Patients enrolled in the combination arm of the Dose Escalation phase should have discontinued that therapy due to either disease progression or reasons other than immune-related toxicity. Patients in all phases of the study may not have received prior IL 2 directed therapy.

Patients will be enrolled in the monotherapy and combination therapy arms of the Dose Expansion Phase who have locally advanced or metastatic renal clear cell carcinoma or locally advanced or metastatic cutaneous malignant melanoma. For monotherapy dose expansion, patients with mRCC may have received up to 3 prior lines of therapy and must have received prior anti-angiogenic therapy (vascular endothelial growth factor inhibitor [VEGFi]) and a CPJ. Patients with BRAF wild-type cutaneous malignant melanoma may have received 1 prior line of therapy and must have received a CPI, anti-PD-(L)1 inhibitor therapy alone or in combination with a cytotoxic T lymphocyte associated protein 4 (CTLA-4) inhibitor or other therapy. Patients with v-raf murine sarcoma viral oncogene homolog B1 (BRAF) mutant melanoma may have received 2 prior lines of therapy, which included a CPI and a BRAF inhibitor with or without a mitogen-activated protein kinase kinase (MEK) inhibitor. For the mRCC patients in the combination part of the Dose Expansion Phase specifically, patients must have had no more than 2 prior lines of therapy, only 1 of which included an anti-PD-(L)1 inhibitor therapy alone or in combination and a VEGFi. Cutaneous malignant melanoma patients enrolled in the combination part of the Dose Expansion Phase may have received 0-2 prior lines of therapy depending upon BRAF mutation status. Patients in the combination therapy arms of the dose expansion phase should not have discontinued prior CPI therapy for immune-related toxicities.

### Inclusion Criteria

Each patient must meet all the following criteria to participate in the study:
1. Able to understand and voluntarily sign a written informed consent form (ICF) and is willing and able to comply with protocol requirements;
2. Has histological or cytological documentation of the solid tumor indication for which an anti PD-(L)1 is indicated (e.g., melanoma, non-small cell lung cancer, small cell lung cancer, head and neck squamous cell cancer, urothelial cancer, microsatellite instability-high or mismatch repair deficient cancer, microsatellite instability high or mismatch repair deficient colorectal cancer, gastric cancer, esophageal cancer, cervical cancer, hepatocellular carcinoma, Merkel cell carcinoma, renal clear cell carcinoma, endometrial carcinoma, tumor mutational burden-high cancer, cutaneous squamous cell carcinoma, advanced basal cell carcinoma) for all parts of the clinical study;
3. Monotherapy Dose Escalation: Patients with relapsed/refractory locally advanced or metastatic solid tumors for which immunotherapy is approved, who have progressed on or are intolerant to standard therapy, including CPIs, or for whom no standard therapy with proven benefit exists;

Combination Dose Escalation: Patients with relapsed/refractory locally advanced or metastatic solid tumors for which immunotherapy is approved, who have progressed on or are intolerant to standard therapy, including CPIs, or for whom no standard therapy with proven benefit exists. Patients must have progressed on prior CPI as defined by RECIST 1.1 or iRECIST or discontinued for reasons other than toxicity.
Patients in either dose escalation arm may have received no more than 3 prior lines of therapy.

Monotherapy Dose Expansion: Patients with relapsed advanced/mRCC or cutaneous malignant melanoma who received prior CPI alone or in combination.
Arm A: Patients with relapsed advanced or mRCC: may have received no more than 3 prior lines of therapy, only 1 of which included CPI, and must have received an anti-angiogenic agent (VEGFi);
Arm B: Patients with relapsed advanced or metastatic cutaneous malignant melanoma: may have received no more than 1 prior line of therapy for BRAF V600 wild type (WT), no more than 2 prior lines of therapy for BRAF V600 mutant, and may have received BRAF inhibitor with or without mitogen-activated protein kinase kinase (MEK) inhibitor. Adjuvant therapy is excluded as 1 of the lines of therapy if there were >6 months to relapse. T-VEC therapy is allowed, but treated lesions cannot be used as target lesions for biopsies.

Combination Dose Expansion: Patients with relapsed advanced/mRCC or metastatic cutaneous malignant melanoma.
Arm C: Patients with relapsed advanced or mRCC: may have received no more than 2 prior lines of therapy, only 1 of which included CPI, and must have received an anti-angiogenic agent (VEGFi);
Arm D: Patients with relapsed advanced or metastatic cutaneous malignant melanoma: may have received 0-2 prior lines of therapy depending upon BRAF status, 0-1 prior lines of therapy for BRAF V600 WT and 0-2 prior lines of therapy for VRAF V600 mutant, which could include a CPI and BRAF inhibitor with or without a MEK inhibitor. Adjuvant therapy is excluded as 1 of the lines of therapy if there were >6 months to relapse. T-VEC therapy is allowed, but treated lesions cannot be used as target lesions or for biopsies.

4. ≥18 years of age;

5. Eastern Cooperative Oncology Group (ECOG) Performance Status of 0 or 1;

6. Has at least 1 measurable lesion per RECIST 1.1 (lesions situated in a previously irradiated area are considered measurable if progression has been demonstrated in such lesions);

7. Agrees to undergo a pre-treatment and post-treatment biopsy of a primary or metastatic solid tumor lesion;

8. Has adequate organ and bone marrow function defined by:

$$Absolute neutrophil count \geq 1.5 \times 1 {0}^{9} / L ( \geq 1500 / {mm}^{3} ) ;$$

b. Hemoglobin≥9.0 g/dL or equivalent. Criteria must be met without packed red blood cell transfusion within the prior 2 weeks;

$$Platelet count \geq 100 \times 1 {0}^{9} / L ( \geq 100 , 000 / {mm}^{3} ) ;$$

d. Total bilirubin≤1.5×ULN in the absence of Gilbert's syndrome and ≤3×ULN if the patient has Gilbert's syndrome;

e. Measured or calculated creatinine clearance (estimated glomerular filtration rate) 30 mL/min/1.73 m²; and

f. ALT and AST≤2.5×ULN or ≤5×ULN for patients with hepatic metastases.

9. Willingness of men and women of reproductive potential to observe highly effective birth control for the duration of treatment and for 4 months following the last dose of study drug;

Male study participants should refrain from sperm donation during study treatment and up to 6 months following the last dose of study drug.

A woman of childbearing potential is a woman who is fertile following menarche and until becoming post-menopausal unless permanently sterile. Permanent sterilization methods include hysterectomy, bilateral salpingectomy, and bilateral oophorectomy.

A man is considered fertile after puberty unless permanently sterile by bilateral orchidectomy.

### Exclusion Criteria

Patients who meet any of the following criteria will be excluded from participating in the study:
1. Have a history of another active malignancy (a second cancer) within the previous 2 years except for localized cancers that are not related to the current cancer being treated, are considered cured, and, in the opinion of the Investigator, presents a low risk of recurrence. These exceptions include, but are not limited to, basal or squamous cell skin cancer, superficial bladder cancer, or carcinoma in situ of the prostate, cervix, or breast;
2. Has a history of (non-infectious) pneumonitis/interstitial lung disease that required steroids or has current pneumonitis/interstitial lung disease.
3. Have a diagnosis of uveal or mucosal melanoma in Dose Expansion Phase arms;
4. Have received prior IL-2-directed therapy;
5. Have had an allogeneic tissue/solid organ transplant;
6. Have known symptomatic brain metastases requiring steroids. Patients with previously diagnosed brain metastases are eligible if they have completed their treatment, have recovered from the acute effects of radiation therapy or surgery prior to enrollment, and are neurologically stable and asymptomatic;
7. Have significant cardiovascular disease, including myocardial infarction, arterial thromboembolism, or cerebrovascular thromboembolism, within 6 months prior to the first dose of study drug; symptomatic dysrhythmias or unstable dysrhythmias requiring medical therapy; angina requiring therapy; symptomatic peripheral vascular disease; New York Heart Association Class 3 or 4 congestive heart failure; or history of congenital prolonged QT syndrome;
8. Have significant electrocardiogram (ECG) abnormalities at Screening, including unstable cardiac arrhythmia requiring medication, left bundle branch block, second-degree atrioventricular (AV) block type II, third-degree AV block, ≥Grade 2 bradycardia, or QT interval corrected for heart rate using Fridericia's formula (QTcF) >470 msec;
9. Have an active autoimmune disease that required systemic treatment in the past 2 years (i.e., with use of disease-modifying antirheumatic agents or immunosuppressive drugs); Note: Replacement therapy (e.g., thyroxine, insulin, or physiologic corticosteroid replacement therapy for adrenal, thyroid, or pituitary insufficiency) is permitted.
10. Diagnosis of immunodeficiency, is on immunosuppressive therapy, or is receiving chronic systemic or enteric steroid therapy (dose >10 mg/day of prednisone or equivalent) or any other form of immunosuppressive therapy within 7 days prior to the first dose of study drug; Note: At Screening and during study participation, patients may be using systemic corticosteroids (dose≤10 mg/day of prednisone or equivalent) or topical, intraocular, intra articular, or inhaled corticosteroids.
11. Major surgery (excluding placement of vascular access) within 2 weeks prior to the first dose of study drug;
12. Investigational agent or anticancer therapy (including chemotherapy, biologic therapy, immunotherapy, anticancer Chinese medicine, or other anticancer herbal remedy) within 5 half-lives or 4 weeks (whichever is shorter) prior to the first dose of study drug. In addition, no concurrent investigational anticancer therapy is permitted;
13. Has received prior radiotherapy within 2 weeks of start of study treatment. Participants must have recovered from all radiation-related toxicities, not require corticosteroids, and not have had radiation pneumonitis. A 1-week washout is permitted for palliative radiation (≤2 weeks of radiotherapy) to non-CNS disease;
14. Any unresolved toxicities from prior therapy greater than National Cancer Institute Common Terminology Criteria for Adverse Events (NCI CTCAE) version 5.0 Grade 1 at the time of starting study drug with the exception of alopecia and Grade 2 prior platinum therapy related neuropathy;
15. Use of a strong inhibitor or inducer of cytochrome P450 (CYP)3A4 prior to starting study drug and during study participation;
16. Use of sensitive substrates to major CYP450 isozymes;
17. Have any illness, medical condition, organ system dysfunction, or social situation, including mental illness or substance abuse, deemed by the Investigator to be likely to interfere with a patient's ability to sign the ICF, adversely affect the patient's ability to cooperate and participate in the study, or compromise the interpretation of study results;
18. Received a live or live-attenuated vaccine within 30 days of the first dose of study drug; Note: Administration of killed vaccines or other formats are allowed.
19. Active, uncontrolled systemic bacterial, viral, or fungal infection;
20. Known human immunodeficiency virus (HIV) antibody;
21. Active infection as determined by hepatitis B surface antigen and hepatitis B core antibody, or hepatitis B virus DNA by quantitative polymerase chain reaction (qPCR) testing;
22. Active infection as determined by hepatitis C virus (HCV) antibody or HCV RNA by qPCR testing;
23. Pregnant or lactating; Note: Defined as a WOCBP who has a positive urine pregnancy test (within 72 hours) prior to treatment. If the urine test is positive or cannot be confirmed as negative, a serum pregnancy test will be required.
24. History of hypersensitivity to any of the study drug components; or
25. Patients will be excluded from the combination arms of the IL-2 prodrug with pembrolizumab if they discontinued prior anti-PD-(L)-1 therapy due to any grade immune-related adverse events (irAEs) except for thyroid abnormalities or adverse events on replacement therapy.

### Study Treatments

### Investigational Product

The IL-2 prodrug has been developed as a lyophilized product supplied in 20R glass vials with flip caps. Upon reconstitution with sterile water, each vial will contain 6 mL nominal volume at 5 mg/mL or 10 mg/mL. The lyophilized IL-2 prodrug will be stored at 2 degrees C. to 8 degrees C.

### Study Drug Administration

### General Dosing Instructions

IL-2 prodrug:

The IL-2 prodrug monotherapy will be administered as a 15-minute IV infusion via syringe pump Q2W in 28 day cycles until progressive disease by RECIST, unacceptable toxicity, withdrawal of consent by the patient, discontinuation of the patient by the Investigator, Sponsor decision to terminate the study or treatment, or death. Infusion duration may be prolonged in the event of an infusion-related reaction.

During the Dose Escalation Phase, a stagger of at least 2 days is required between dosing of the first and second patients at each new untested dose level, and a stagger of 1 day is required between dosing of the second and third patients.

### Pembrolizumab:

Pembrolizumab will be administered using IV infusion on Day 1 of each 6-week (42-day) treatment cycle after all procedures and assessments have been completed.

Pembrolizumab will be administered as a dose of 400 mg using a 30-minute IV infusion. Sites should make every effort to target infusion timing to be as close to 30 minutes as possible; however, given the variability of infusion pumps from site to site, a window between −5 minutes and +10 minutes is permitted (i.e., infusion time is 30 minutes [−5 minutes/+10 minutes]).

For the IL-2 prodrug+pembrolizumab combination arms of the Dose Escalation Phase or Dose Expansion Phase, pembrolizumab will be administered first. Patients can receive IL-2 prodrug infusion approximately 30 minutes after they have received the entire infusion of pembrolizumab.

Pembrolizumab dosing will be capped at 18 cycles (˜2 years). However, the IL-2 prodrug may be continued as a monotherapy for as long as the patient is deriving clinical benefit and per criteria in the IL-2 prodrug section above.

### Dose Modification Guidelines

If a patient experiences treatment-emergent adverse events (TEAEs) that may be related to the IL-2 prodrug, pembrolizumab, or both, including irAEs, further doses might be modified, halted, or permanently discontinued. During Cycle 1, dose modification should be avoided if a patient has not experienced a≥Grade 2 TEAE related to study drug. In subsequent cycles, dose interruptions and/or modifications are permissible.

### Study Procedures

### Tests and Evaluations

### Vital Signs and Physical Examinations

Vital signs will be measured and will include measurements of systolic and diastolic blood pressure, heart rate, and body temperature.

A physical examination and review of relevant systems, body weight, and height will occur at screening. Height will be measured at the Screening Visit only but may be measured later if missed. An abbreviated physical examination that is directed by disease site and symptoms will be performed on Day 1 of subsequent cycles after Cycle 1.

### Pharmacokinetics and Electrocardiograms

### Pharmacokinetics

Plasma samples for IL-2 prodrug PK assessment of the parent compound and free IL-2 will be obtained. Additional PK assessments may be conducted when considered necessary by the Investigator to understand exposure in relationship to possible safety or anti-tumor activity findings. Samples will be collected, and concentrations of the IL-2 prodrug and free IL-2 will be determined with a validated bioanalytical method.

### Electrocardiograms

Twelve-lead ECGs will be performed. Serial triplicate 12-lead ECGs (separated by ≥1 minute) will be performed throughout the DLT period during the Dose Escalation Phase. Single 12-lead ECGs will be performed after the DLT period during the Dose Escalation Phase and throughout the Dose Expansion Phase. Single ECGs should be repeated if an anomaly or abnormality is observed. When the ECG measurements coincide with blood sample draws, the ECG assessment should be timed sufficiently prior to blood sample collection to not impact the PK sample collection time. ECGs will be recorded after the patient has been in a resting (semi recumbent or semi-supine) position breathing quietly for 5 minutes. All pre-dose ECGs will be performed 15 to 30 minutes prior to study drug administration. The evaluation of ECGs will include assessment of changes in the following ECG parameters: heart rate, PR, QRS, QT, and QTcF intervals. Medpace Core Laboratory will provide equipment for the Dose Escalation Phase of the study and ECG images will be stored centrally. Local evaluation and equipment for ECGs will be utilized during the Expansion Phase of the study, and ECG images will be stored locally.

### Pharmacodynamics

Whole blood samples will be collected for pharmacodynamic assessments and biomarker assessments, as specified in Table 8 below.

While the goal of the biomarker assessments is to provide supportive data for the clinical study, there may be circumstances when a decision is made to stop a collection, not perform, or discontinue an analysis due to either practical or strategic reasons (e.g., inadequate sample number, issues related to the quality of the sample or issues related to the assay that preclude analysis, impossibility to perform correlative analyses, etc.). Therefore, depending on the results obtained during the study, sample collection analysis may be omitted at the discretion of the Sponsor.

TABLE 8
Biomarker Sample Collection Plan for the IL-2 prodrug

| | Visit/Time | Approx. | | |
|---|---|---|---|---|
| Sample Type | point | volume | Marker | Purpose |
| Newly obtained | Baseline | 5-6 passes of | Immunophenotype | To characterize |
| tumor biopsy | | a core needle | Immune cell | changes in tumor |
| *Archival tumor is | | biopsy | characterization: | associated |
| acceptable at | | | changes in gene | immune cells, |
| baseline if obtaining | | | expression | including T cell |
| a fresh biopsy is | | | Immune cell | subsets. |
| medically | | | characterization: | Functional |
| unfeasible. In these | | | changes in immune | characterization |
| cases, | | | contexture | of tumor |
| immunophenoyping | | | | immune |
| will not be | | | | infiltrates, |
| performed | | | | cytotoxicity and |
| | | | | effector |
| | | | | functions. |
| Newly obtained | C1D22-D28 | 5-6 passes of | | To visualize |
| tumor biopsy | | a core needle | | changes in |
| | | biopsy | | cellular |
| | | | | composition of |
| | | | | tumor immune |
| | | | | infiltrates and |
| | | | | PD-L1 |
| | | | | expression |
| Blood (Serum or | C1D1 pre- | | IL-2Ra (sCD25) | To investigate |
| Plasma) | dose | | Soluble cytokines | target |
| | C1D2 | | | engagement |
| | C1D3 | | | and assess |
| | | | | systemic |
| | | | | cytokine |
| | | | | modulation |
| Blood | C1D1 pre- | | Lymphocyte/Eosinophil | Indicator of |
| | dose | | levels | target |
| | C1D2 | | | engagement |
| | C1D8 | | | |
| Blood (collected in | Baseline, | 2 ml | Immunophenotype | To characterize |
| cytochex tube) | C1D8 | | | changes in |
| | | | | peripheral |
| | | | | immune cells, |
| | | | | including T cell |
| | | | | subsets |

Tumor Measurements per RECIST and iRECIST

Tumors will be assessed based on RECIST 1.1 (see Tumor Measurements and Assessment of Disease Response using RECIST 1.1) and iRECIST (see Modified Response Evaluation Criteria in Solid Tumors 1.1 for Immune-Based Therapeutics (IRECIST) Quick Reference). Baseline disease assessment will include radiographic tumor measurements using computed tomography (CT) or magnetic resonance (MR) imaging of the chest, abdomen, pelvis, or any other areas with suspected disease involvement at Screening within 28 days of Cycle 1 Day 1. Patients with CNS metastases must have brain imaging (MR imaging preferred, CT with contrast is acceptable if MR imaging contraindicated) during Screening. For each modality, intravenous (IV) and oral contrast should be utilized (chest CT does not require IV contrast) unless there is a clear contraindication (e.g., decreased renal function or allergy that cannot be addressed with standard prophylactic treatments). On-study scans should be performed every 8 weeks (+7 days) from Cycle 1 Day 1 for the first 6 cycles and every 12 weeks (+7 days) thereafter, including imaging of the chest, abdomen, pelvis, or any other areas of known disease at baseline, using the same modality as used for baseline imaging until progressive disease per RECIST 1.1 and iRECIST, withdrawal of consent, or initiation of a new anticancer therapy.

### Biopsied and Archived Tumor Samples

Pre- and post-treatment biopsies are required for immunophenotyping, cancer gene expression analysis, and tumor immune contexture assessment by immunohistochemistry. However, in the event that collection of a fresh biopsy with required number of cores are medically infeasible, patient may continue on study and an archival tumor sample will be requested. At baseline, a fresh biopsy (2 cores) is required for immunophenotyping by flow cytometry. Also at baseline, tissue from either a newly obtained (4 cores) or archival biopsy, if fresh biopsy is medically infeasible (tumor block preferred), will be collected for cancer gene expression analysis and tumor immune contexture assessment by immunohistochemistry. The baseline sample may be collected any time after enrollment during the 28-day Screening period. On study, a biopsy (6 cores) will be collected between Day 22 and Day 28 of Cycle 1 for immunophenotyping, cancer gene expression analysis and tumor immune contexture assessment by immunohistochemistry. The timing of the second biopsy may shift based on evolving biomarker data.

### Efficacy and Pharmacokinetic Assessments

### Study Endpoints

### Primary Endpoints (Dose Escalation Phase)

The primary endpoints of the Dose Escalation Phase of this study are the following:
MTD and/or RDE for the IL-2 prodrug alone;
MTD and/or RDE for the IL-2 prodrug in combination with pembrolizumab; and
Frequency, severity, and relatedness of TEAEs and serious adverse events (SAEs), changes in safety laboratory parameters, and DLTs (if observed) for the IL-2 prodrug alone and in combination with pembrolizumab.

### Primary Endpoints (Dose Expansion Phase)

The primary endpoints of the Dose Expansion Phase of this study are the following:
Frequency, severity, and relatedness of TEAEs and SAEs, changes in safety laboratory parameters, and DLTs (if observed) for the IL-2 prodrug alone and in combination with pembrolizumab; and
ORR(ORR=CR+PR), DOR by RECIST 1.1 and iORR (iORR=iCR+iPR) by iRECIST.

### Secondary Endpoints (Dose Escalation and Dose Expansion Phases)

The secondary endpoints of the Dose Escalation and Dose Expansion Phases of this study are the following:
Plasma concentrations of IL-2 prodrug and free IL-2 and calculated PK parameters;
DOR, and PFS by RECIST 1.1 and iDOR, and iPFS by iRECIST;
Characterization of changes in peripheral immune cells, including T cell subsets, from baseline in response to IL-2 prodrug alone, and in combination with pembrolizumab;
Changes in immunological biomarkers in baseline and post-treatment tumor biopsies in response to IL-2 prodrug alone, and in combination with pembrolizumab, as determined by immunohistochemistry (IHC); and
Incidence of immunogenicity of IL-2 prodrug (alone and in combination with pembrolizumab), as indicated by levels of ADAs.

### EXPLORATORY ENDPOINTS

The exploratory endpoints of the Dose Escalation and Dose Expansion Phases of this study are the following:
PD effects of the IL-2 prodrug alone, and in combination with pembrolizumab, as follows:
   Modulation of cytokines including, but not limited to, IL-2, IL-4, IL-5, IL-6, IL-8, IL-10, IL-13, IL-15, soluble CD25, interferon gamma, transforming growth factor beta, tumor necrosis factor alpha
   Changes in levels of lymphocytes and eosinophils in peripheral blood;
   Characterization of intra-tumoral immune cells, including T-cell subsets;
   Changes in gene expression profiles of immune response in tumor;
   Changes in intra-tumoral levels of IL-2.

### Safety Assessments: Adverse Events of Special Interest

The safety and tolerability profile of IL-2 prodrug, alone and in combination with a pembrolizumab, will be assessed by monitoring adverse events (including DLTs, SAEs, and adverse events of special interest [AESIs]), physical examination findings (including ECOG performance status), clinical laboratory evaluations, vital sign measurements, and ECGs. Adverse events will be graded according to the NCI CTCAE version 5.0. For this study, AESIs include the following:
≥Grade 2 CRS; and
≥Grade 2 CLS.

For combination arms, AESIs include the following:
an overdose of pembrolizumab, as defined in Treatment of Overdose, that is not associated with clinical symptoms or abnormal laboratory results.
an elevated AST or ALT lab value that is greater than or equal to 3×the upper limit of normal and an elevated total bilirubin lab value that is greater than or equal to 2×the upper limit of normal and, at the same time, an alkaline phosphatase lab value that is less than 2×the upper limit of normal, as determined by way of protocol-specified laboratory testing or unscheduled laboratory testing.

### Statistical Analysis

Plasma concentrations of IL-2 prodrug and free IL-2 will be determined with a validated bioanalytical assay. The following PK parameters for IL-2 prodrug and free IL-2 will be calculated from plasma concentrations using noncompartmental analyses: maximum observed plasma concentration (Cₘₐₓ), time to maximum observed plasma concentration (Tₘₐₓ), area under the concentration versus time curve from time 0 to t (AUC₀₋₄), area under the concentration time versus curve from time 0 to infinity (AUC_{0-inf}), clearance (CL), volume of distribution (V_{d}), and terminal-elimination half-life (t_{1/2}). Summary statistics will be generated by dose cohort as appropriate.

Plots of mean IL-2 prodrug and free IL-2 plasma concentrations versus time will be generated by dose group and phase in linear and semi-logarithmic form. Individual plasma concentrations versus time graphs will also be provided.

The anti-tumor activity analysis will be conducted on the Safety Analysis Set unless otherwise specified. Tumor response data per RECIST 1.1 and iRECIST criteria will be listed and summarized. ORR will be estimated for each cohort based on the observed proportion of patients whose best overall response is confirmed CR or PR. iORR will be estimated for each cohort based on the observed proportion of patients whose best overall response is confirmed iCR or iPR. The DOR, iDOR, PFS, and iPFS will be summarized descriptively using the Kaplan-Meier method.

In general, all safety analyses will be descriptive and will be presented in tabular format with the appropriate summary statistics for the Safety Analysis Set.

The safety and tolerability profile of IL-2 prodrug, alone and in combination with pembrolizumab, will be assessed by monitoring adverse events (including DLTs, SAEs, and AESIs), physical examination findings (including ECOG performance status), clinical laboratory evaluations, vital sign measurements, and ECGs. Adverse events will be graded according to the NCI CTCAE version 5.0.

The number and percentage of patients with TEAEs will be summarized by System Organ Class and preferred term. Grade 3 or higher TEAEs, drug-related TEAEs, treatment-emergent SAEs, drug-related treatment-emergent SAEs, TEAEs leading to discontinuation of study drug, drug-related TEAEs leading to discontinuation of study drug, TEAEs leading to dose reduction/interruption of study drug, and drug-related TEAEs leading to dose reduction/interruption of study drug will be summarized in the same manner. DLTs during Cycle 1 in the Dose Escalation Phase of the study will be summarized by primary System Organ Class and preferred term.

### Analysis of Anti-Tumor Activity

All efficacy will be done using RECIST 1.1 and iRECIST criteria based on local investigator assessment.

### Overall Response Rate:

Overall response rate as defined by achieving confirmed CR and/or PR will be presented by percentage rates and 95% confidence intervals (CIs). For changes in solid tumor size, waterfall plots will be presented. For all response assessments, swimmers plots will be presented. All response assessments will be listed.

### Clinical Benefit Rate at Months 3, 6, and 9:

Clinical benefit rate at Months 3, 6, and 9, as defined by achieving CR and/or PR and/or SD, will be presented by percentage rates and 95% CIs. Waterfall plots will be presented.

### Duration of Response:

The duration of response defined as time from first assessment of PR or CR to follow-on first assessment of PD will be summarized by descriptive statistics including median duration of response and respective 95% CIs. Duration of response will also be listed.

### Progression Free Survival (PFS) and Overall Survival (OS):

Time from first treatment received until PD/OS will be summarized by Kaplan-Meier estimates, median PFS/OS and respective 95% CIs. Patients with no event will be censored at the last available tumor assessment for PFS and at the last timepoint known alive for OS.

iORR will be estimated for each cohort based on the observed proportion of patients whose best overall response is confirmed iCR or iPR. The DOR, iDOR, PFS, and iPFS will be summarized descriptively using the Kaplan-Meier method.

### Analysis of Pharmacokinetics

The PK Analysis Set will be used for summaries of all PK data. No formal statistical analysis beyond descriptive statistics is planned. For each PK parameter, individual and mean data and summary statistics (including number of patients, arithmetic mean, geometric mean (for time to maximum plasma concentration (tₘₐₓ) and time to last measurable plasma concentration (tₗₐₛₜ) no geometric mean will be calculated), standard deviation (StD), confidence value (CV), median, Min and Max) will be presented.

### Analysis of Pharmacodynamics

The result, change, percent change, and maximum percent change in immunologic changes to serum cytokines and immune cell subsets in the blood and tumor microenvironment will be summarized descriptively.

### Sample Size Determination

The overall sample size for this study is approximately 150 patients between the Dose Escalation Phase and Dose Expansion Phase and will depend on the observed DLT profiles of IL-2 prodrug monotherapy and IL-2 prodrug in combination with pembrolizumab.

### Example 3: CT26 Mouse Model

The CT26 cell line, a rapidly growing colon adenocarcinoma cell line that expresses MMP9 in vitro, was used. Using this tumor model, the ability of fusion proteins to affect tumor growth was examined.

TABLE 9

| | | | Inducible IL-2 | | Inducible IL-2 | |
|---|---|---|---|---|---|---|
| | | | prodrug | | prodrug | |
| | | | (Compound 1) | Anti-PD1 | (Compound 1) | Anti-PD1 |
| Gr. | N | Treatment | dose | Dose | schedule | schedule |
| 1^{#} | 8 | vehicle | na | na | na | na |
| 2 | 8 | inducible IL-2 | 100 μg/animal | na | Days 1 and 8 | na |
| | | prodrug | | | | |
| 3 | 8 | Anti-PD-1 | na | 200 μg/animal | na | Days 1, 4, 8 and 11 |
| 4 | 8 | inducible IL-2 | 100 μg/animal | 200 μg/animal | Days 1 and 8 | Days 1, 4, 8 and 11 |
| | | prodrug + | | | | |
| | | anti-PD-1 | | | | |

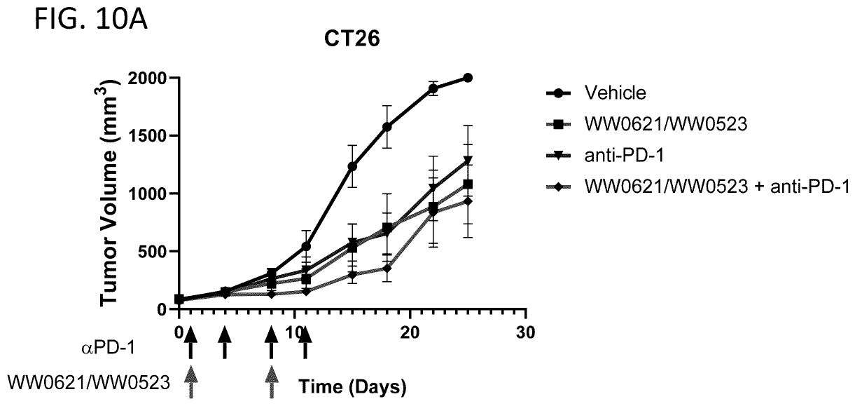

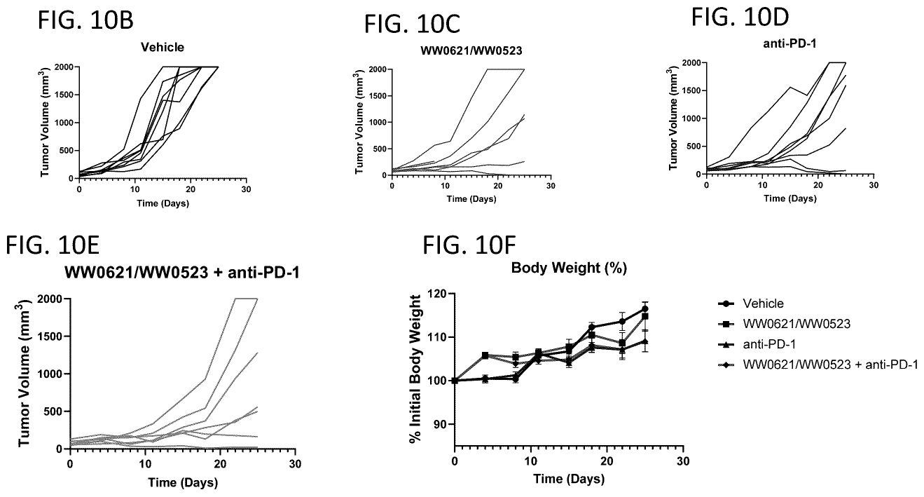

Mice were anaesthetized with isoflurane for implant of cells to reduce the ulcerations. Female BALB/c mice were set up with 1.5×10⁵ CT26 tumor cells se in flank. Cell Injection Volume was 0.1 mL/mouse. Mouse age at start date was 8 to 12 weeks. Pair matches were performed when tumors reached an average size of 100-150 mm³ and treatment began according to Table 9. The inducible IL-2 prodrug was Compound 1 (WW0621/WW0523), and the anti-PD-1 antibody was RMP1-14. Body weights were taken at initiation and then biweekly to the end of the study. Caliper measurements of tumor size were taken biweekly to the end of the study. Any adverse reactions were reported immediately. Any individual animal with a single observation of ≥than 30% body weight loss or three consecutive measurements of ≥25% body weight loss was euthanized. Any group with a mean body weight loss of >20% or >10% mortality resulted in stopped dosing; the group was not euthanized, and recovery was allowed. Animals were monitored individually. The endpoint of the study was a tumor volume of 1500 mm³ or 45 days, whichever comes first. Responders were followed longer. When the endpoint was reached, the animals were euthanized. Treatment with Compound 1 or anti-PD-1 in this study showed efficacy as a monotherapy. The combination therapy using Compound 1 and an anti-PD1 antibody improved tumor control, and was more effective than either Compound 1 or an anti-PD-1 antibody monotherapy. Results are shown in FIGS. 10A-10F.

### OTHER EMBODIMENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

1. A method for treating cancer, comprising administering to a subject in need thereof a combination therapy comprising Compound 1 (SEQ ID NO:1/SEQ ID NO:5), Compound 2 (SEQ ID NO:2/SEQ ID NO:5), Compound 3 (SEQ ID NO:3/SEQ ID NO:5), Compound 4 (SEQ ID NO:4/SEQ ID NO:5) or an amino acid sequence variant of any of the foregoing and an anti-PD-1 antibody, or antigen binding fragment thereof; wherein the anti-PD-1 antibody, or antigen binding fragment thereof, comprises: (a) light chain CDRs SEQ ID NOs: 6, 7 and 8 and (b) heavy chain CDRs SEQ ID NOs: 11, 12 and 13.

2. The method of claim 1, wherein a) Compound 1 comprises a first polypeptide chain of SEQ ID NO:1 and a second polypeptide chain of SEQ ID NO:5, and the amino acid sequence variant of Compound 1 comprises a first polypeptide chain that has at least about 80% identity to SEQ ID NO:1 and a second polypeptide chain that has at least about 80% identity to SEQ ID NO:5, b) Compound 2 comprises a first polypeptide chain of SEQ ID NO:2 and a second polypeptide chain of SEQ ID NO:5, and the amino acid sequence variant of Compound 2 comprises a first polypeptide chain that has at least about 80% identity to SEQ ID NO:2 and a second polypeptide chain that has at least about 80% identity to SEQ ID NO:5, c) Compound 3 comprises a first polypeptide chain of SEQ ID NO:3 and a second polypeptide chain of SEQ ID NO:5, and the amino acid sequence variant of Compound 3 comprises a first polypeptide chain that has at least about 80% identity to SEQ ID NO:3 and a second polypeptide chain that has at least about 80% identity to SEQ ID NO:5, and d) Compound 4 comprises a first polypeptide chain of SEQ ID NO:1 and a second polypeptide chain of SEQ ID NO:4, and the amino acid sequence variant of Compound 4 comprises a first polypeptide chain that has at least about 80% identity to SEQ ID NO:4 and a second polypeptide chain that has at least about 80% identity to SEQ ID NO:5.

3. The method of claim 1 or 2, wherein the anti-PD-1 antibody, or antigen binding fragment thereof, comprises (a) a heavy chain variable region comprising SEQ ID NO:14 or a variant thereof, and (b) a light chain variable region comprising SEQ ID NO:9 or a variant thereof.

4. The method of claim 1 or 2, comprising the anti-PD-1 antibody that comprises a heavy chain and a light chain, and wherein the heavy and light chains comprise the amino acid sequences in SEQ ID NO:10 and SEQ ID NO:5, respectively

5. The method of claim 1 or 2, comprising the anti-PD-1 antibody that is pembrolizumab or a pembrolizumab variant.

6. The method of claim 1 or 2, comprising the anti-PD-1 antibody that is pembrolizumab (or a biosimilar).

7. The method of any one of the preceding claims, wherein an effective amount of the combination therapy is administered to the subject.

8. The method of any one of the preceding claims, wherein Compound 1, Compound 2, Compound 3, Compound 4 or an amino acid sequence variant of any of the foregoing is administered concurrently with the anti-PD-1 antibody, or antigen binding fragment thereof.

9. The method of claim 8, wherein the anti-PD-1 antibody, or antigen binding fragment thereof, is administered prior to the administration of Compound 1, Compound 2, Compound 3, Compound 4 or an amino acid sequence variant of any of the foregoing is administered.

10. The method of claim 8, wherein the anti-PD-1 antibody, or antigen binding fragment thereof, is administered after to the administration of Compound 1, Compound 2, Compound 3, Compound 4 or an amino acid sequence variant of any of the foregoing is administered.

11. The method of claim 8, wherein the anti-PD-1 antibody, or antigen binding fragment thereof, is administered, and then about 30 minutes following completion of the administration of the anti-PD-1 antibody, or antigen binding fragment thereof, Compound 1, Compound 2, Compound 3, Compound 4 or an amino acid sequence variant of any of the foregoing is administered.

12. The method of claim 8, wherein Compound 1, Compound 2, Compound 3, Compound 4 or an amino acid sequence variant of any of the foregoing is administered, and then about 30 minutes following completion of the administration of Compound 1, Compound 2, Compound 3, Compound 4 or an amino acid sequence variant of any of the foregoing, the anti-PD-1 antibody, or antigen binding fragment thereof, is administered.

13. The method of any one of the preceding claims, wherein Compound 1, Compound 2, Compound 3, Compound 4 or an amino acid sequence variant of any of the foregoing and the anti-PD-1 antibody, or antigen binding fragment thereof, are administered intravenously.

14. The method of claim 13, wherein the administration is by intravenous infusion.

15. The method of any one of claims 1-12, wherein the administration is oral, parenteral, intravenous, intra-articular, intraperitoneal, intramuscular, subcutaneous, intracavity, transdermal, intrahepatical, intracranial, nebulization/inhalation, by installation via bronchoscopy, or intratumoral.

16. The method of any one of the preceding claims, wherein about 100 mg to about 600 mg of the anti-PD-1 antibody, or antigen binding fragment thereof, is administered about every three to six weeks.

17. The method of claim 16, wherein 200 mg of the anti-PD-1 antibody, or antigen binding fragment thereof, is administered about every three weeks.

18. The method of claim 16, wherein 400 mg of the anti-PD-1 antibody, or antigen binding fragment thereof, is administered about every six weeks.

19. The method of any one of the preceding claims, wherein about 1 mg to about 500 mg of Compound 1, Compound 2, Compound 3, Compound 4 or an amino acid sequence variant of any of the foregoing is administered every two weeks.

20. The method of claim 19, wherein about 1 mg to about 240 mg of Compound 1, Compound 2, Compound 3, Compound 4 or an amino acid sequence variant of any of the foregoing is administered every two weeks.

21. The method of claim 19, wherein about 1 mg, about 3 mg, about 10 mg, about 30 mg, about 60 mg, about 120 mg or about 240 mg of Compound 1, Compound 2, Compound 3, Compound 4 or an amino acid sequence variant of any of the foregoing is administered every two weeks.

22. The method of any one of claims 1-21, wherein the combination is administered as a first line therapy.

23. The method of any one of claims 1-21, wherein the subject has failed to achieve a complete response to a prior treatment or to an ongoing treatment.

24. The method of claim 23, wherein the prior or ongoing treatment comprises treatment with a checkpoint inhibitor.

25. The method of claim 24, wherein the checkpoint inhibitor is an anti-PD-1 antibody or an anti-PD-L1 antibody.

26. The method of claim 24, wherein the checkpoint inhibitor is an anti-CTLA-4 antibody.

27. The method of any one of claims 1-20, wherein the cancer is metastatic.

28. The method of any one of claims 1-27, wherein the cancer comprises a solid tumor.

29. The method of any one of claim 1-27, wherein cancer is a sarcoma or carcinoma.

30. The method of any one of claims 1-26, wherein the cancer is adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

31. The method of any one of claims 1-26 wherein the cancer is colon cancer, lung cancer, melanoma, renal cell carcinoma, or breast cancer.

32. The method of any one of claims 1-26, wherein the cancer is melanoma, non-small cell lung cancer (NSCLC), small cell lung cancer (SCLC), head and neck squamous cell cancer (HNSCC), classical Hodgkin lymphoma (cHL), primary mediastinal large B cell lymphoma (PMBCL), urothelial carcinoma, microsatellite instability high or mismatch repair deficient cancer, microsatellite instability high or mismatch repair deficient colorectal cancer, gastric cancer, esophageal cancer, cervical cancer, hepatocellular carcinoma (HCC), merkel cell carcinoma (MCC), renal cell carcinoma (RCC), endometrial carcinoma, tumor mutational burden high cancer, cutaneous squamous cell carcinoma (cSCC), triple negative breast cancer (TNBC), or oesophageal carcinoma.

33. The method of any one of claims 1-26, wherein the cancer is metastatic renal clear cell carcinoma or metastatic cutaneous malignant melanoma.

34. The method of any one of claims 1-26, wherein the cancer is non-small cell lung cancer, and the method further comprises administering to the subject pemetrexed and a platinum chemotherapeutic agent.

35. The method of any one of claims 1-26, wherein the cancer is non-small cell lung cancer, and the method further comprises administering to the subject paclitaxel.

36. The method of any one of claims 1-26, wherein the cancer is non-small cell lung cancer, and the method further comprises administering to the subject protein bound paclitaxel.

37. The method of any one of claims 1-26, wherein the cancer is head and neck squamous cell cancer, and the method further comprises administering to the subject fluorouracil.

38. The method of any one of claims 1-26, wherein the cancer is esophageal or gastroesophageal junction carcinoma, and the method further comprises administering to the subject a platinum- and fluoropyrimidine-based chemotherapeutic agent.

39. The method of any one of claims 1-26, wherein the cancer is renal cell carcinoma, and the method further comprises administering to the subject axitinib.

40. The method of any one of claims 1-26, wherein the cancer is endometrial carcinoma, and the method further comprises administering to the subject lenvatinib.

41. The method of any one of claims 1-26, wherein the cancer is triple-negative breast cancer, and the method further comprises administering to the subject a chemotherapeutic agent.

42. A pharmaceutical composition comprising Compound 1, Compound 2, Compound 3, Compound 4, or an amino acid sequence variant of any of the foregoing, for use in combination with an anti-PD-1 antibody, or antigen binding fragment thereof, for treating cancer in a subject in need thereof according to the method of any one of claims 1-41; wherein the anti-PD-1 antibody, or antigen binding fragment thereof, comprises: (a) light chain CDRs SEQ ID NOs: 6, 7 and 8 and (b) heavy chain CDRs SEQ ID NOs: 11, 12 and 13.

43. A pharmaceutical composition comprising an anti-PD-1 antibody, or antigen binding fragment thereof, for use in combination with Compound 1, Compound 2, Compound 3, Compound 4, or an amino acid sequence variant of any of the foregoing, for treating cancer in a subject in need thereof according to the method of any one of claims 1-41; wherein the anti-PD-1 antibody, or antigen binding fragment thereof, comprises: (a) light chain CDRs SEQ ID NOs: 6, 7 and 8 and (b) heavy chain CDRs SEQ ID NOs: 11, 12 and 13.

44. A pharmaceutical composition comprising Compound 1, Compound 2, Compound 3, Compound 4 or an amino acid sequence variant of any of the foregoing thereof and an anti-PD-1 antibody, or antigen binding fragment thereof, and a suitable carrier; wherein the anti-PD-1 antibody, or antigen binding fragment thereof, comprises: (a) light chain CDRs SEQ ID NOs: 6, 7 and 8 and (b) heavy chain CDRs SEQ ID NOs: 11, 12 and 13.

45. The pharmaceutical composition of claim 44, wherein a) Compound 1 comprises a first polypeptide chain of SEQ ID NO:1 and a second polypeptide chain of SEQ ID NO:5, and the amino acid sequence variant of Compound 1 comprises a first polypeptide chain that has at least about 80% identity to SEQ ID NO:1 and a second polypeptide chain that has at least about 80% identity to SEQ ID NO:5, b) Compound 2 comprises a first polypeptide chain of SEQ ID NO:2 and a second polypeptide chain of SEQ ID NO:5, and the amino acid sequence variant of Compound 2 comprises a first polypeptide chain that has at least about 80% identity to SEQ ID NO:2 and a second polypeptide chain that has at least about 80% identity to SEQ ID NO:5, C) Compound 3 comprises a first polypeptide chain of SEQ ID NO:3 and a second polypeptide chain of SEQ ID NO:5, and the amino acid sequence variant of Compound 3 comprises a first polypeptide chain that has at least about 80% identity to SEQ ID NO:3 and a second polypeptide chain that has at least about 80% identity to SEQ ID NO:5, and c) Compound 4 comprises a first polypeptide chain of SEQ ID NO:1 and a second polypeptide chain of SEQ ID NO:4, and the amino acid sequence variant of Compound 4 comprises a first polypeptide chain that has at least about 80% identity to SEQ ID NO:4 and a second polypeptide chain that has at least about 80% identity to SEQ ID NO:5.

46. The pharmaceutical composition of claim 44 or 45, wherein the anti-PD-1 antibody, or antigen binding fragment thereof, comprises (a) a heavy chain variable region comprising SEQ ID NO:14 or a variant thereof, and (b) a light chain variable region comprising SEQ ID NO:9 or a variant thereof.

47. The pharmaceutical composition of claim 44 or 45, comprising the anti-PD-1 antibody that comprises a heavy chain and a light chain, and wherein the heavy and light chains comprise the amino acid sequences in SEQ ID NO:10 and SEQ ID NO:5, respectively.

48. The pharmaceutical composition of claim 44 or 45, comprising the anti-PD-1 antibody that is pembrolizumab or a pembrolizumab variant.

49. The pharmaceutical composition of claim 44 or 45, comprising the anti-PD-1 antibody that is pembrolizumab (or a biosimilar).

50. The pharmaceutical composition of any one of claim 44-49, wherein the composition is a liquid composition for intravenous administration.

51. The pharmaceutical composition of any one of claim 44-49, wherein the composition is a lyophilized composition.

52. The pharmaceutical composition of claim 51, wherein the lyophilized composition is for reconstitution using water for injection or saline, and the reconstituted formulation is suitable of intravenous administration.

53. Compound 1, Compound 2, Compound 3, Compound 4 or an amino acid sequence variant of any of the foregoing and an anti-PD-1 antibody, or antigen binding fragment thereof for use in the treatment of a cancer, wherein the anti-PD-1 antibody, or antigen binding fragment thereof, comprises: (a) light chain CDRs SEQ ID NOs: 6, 7 and 8 and (b) heavy chain CDRs SEQ ID NOs: 11, 12 and 13.


SEQUENCE LISTING (15 sequences)

SEQ ID NO: 1 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 2 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 3 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 4 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 5 - protein, 214 aa, synthetic construct
DIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 15 aa, synthetic construct
RASKGVSTSGYSYLH

SEQ ID NO: 7 - protein, 7 aa, synthetic construct
LASYLES

SEQ ID NO: 8 - protein, 9 aa, synthetic construct
QHSRDLPLT

SEQ ID NO: 9 - protein, 111 aa, synthetic construct [sequence omitted]

SEQ ID NO: 10 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
NYYMY

SEQ ID NO: 12 - protein, 17 aa, synthetic construct
GINPSNGGTNFNEKFKN

SEQ ID NO: 13 - protein, 11 aa, synthetic construct
RDYRFDMGFDY

SEQ ID NO: 14 - protein, 120 aa, synthetic construct [sequence omitted]

SEQ ID NO: 15 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK